(19) United States
(12) Patent Application Publication
(10) Pub. No.: US 2011/0171219 A1
(43) Pub. Date: Jul. 14, 2011

(54) TREATING CANCER STEM CELLS USING TARGETED CARGO PROTEINS

(76) Inventor: Fahar Merchant, West Vancouver (CA)

(21) Appl. No.: 13/119,426

(22) PCT Filed: Sep. 21, 2009
(86) PCT No.: PCT/CA2009/001323

§ 371 Date: Mar. 16, 2011

Related U.S. Application Data
(60) Provisional application No. 61/098,634, filed on Sep. 19, 2008.

(51) Int. Cl. A61K 39/395; A61K 38/16; A61P 35/00

(52) U.S. Cl. 424/134.1; 514/19.3

(57) ABSTRACT

The disclosure provides targeted cargo proteins that are useful for targeting cancer stem cells, and methods of their use in treating cancer.

### CROSS-REFERENCE TO RELATED APPLICATION

This application claim priority to U.S. Provisional Application No. 61/098,634 filed Sep. 19, 2008, herein incorporated by reference.

### FIELD

The disclosure provides targeted cargo proteins that are useful for targeting cancer stem cells, and methods for their use in treating cancer.

### BACKGROUND

Cancer stem cells are a subpopulation of a tumor that have the capacity to self-renew, and thus can give rise to progeny with similar properties. The cancer stem cells are generally slow-growing and are not responsive to traditional anti-cancer therapies targeted to fast-growing cells. Therefore, traditional cancer therapies are likely to inhibit the bulk tumor population but not cancer stem cells, leaving the cancer stem cells intact and able to give rise to more tumor growth. Consequently, cancer may recur as the result of cancer stem cell-driven expansion. Therapies that selectively target bulk tumor cells and cancer stem cells offer another method of treating cancer patients.

### SUMMARY

The disclosure describes proteins and other moieties that target cancer stem cells using a targeting moiety that is linked to a protein or other toxic agent that kills cancer stem cells or inhibits cancer stem cell growth. The protein or other toxic agent that inhibits cancer stem cell growth is referred to as a cargo moiety and the cargo moiety linked to the targeting moiety is collectively referred to as a targeted cargo protein. Targeted cargo proteins are useful, among other things, for treating subjects with cancer, including subjects that display cancers that are recurrent. Therefore, the disclosure provides methods of using the targeted cargo proteins to treat cancer and cancer stem cells in a mammalian subject, such as a human.

The foregoing and other objects and features will become more apparent from the following detailed description.

### BRIEF DESCRIPTION OF THE DRAWING

FIG. 1 shows a schematic representation of the structure and amino acid sequence of an exemplary targeted cargo protein, a circularly permuted IL-4 Pseudomonas toxin, PRX321 (SEQ ID NO: 1). Disulfide bonds are indicated on the drawing.

### SEQUENCE LISTING

The amino acid sequence listed in the accompanying sequence listing is shown using standard three letter code for amino acids, as defined in 37 C.F.R. §1.822. In the accompanying sequence listing:

SEQ ID NO: 1 shows the amino acid sequence of an exemplary targeted cargo protein.

### DETAILED DESCRIPTION

### I. Abbreviations and Terms

PA Proaerolysin

BAD BCL2-associated agonist of cell death

BAX BCL2-associated X protein

EGF Epidermal growth factor

EpCAM Epithelial protein cell adhesion molecule

GMCSF Granulocyte-macrophage colony-stimulating factor

IL-2 Interleukin-2

IL-3 Interleukin-3

IL-4 Interleukin-4

IL-5 Interleukin-5

IL-10 Interleukin-10

IL-13 Interleukin-13

PSMA Prostate specific membrane antigen

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a targeted cargo protein” includes single or plural targeted cargo proteins and is considered equivalent to the phrase “comprising at least about one targeted cargo protein.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs.

Accession Numbers: Reference numbers assigned to various nucleic acid and amino acid sequences in the NCBI database (National Center for Biotechnology Information) that is maintained by the National Institute of Health, U.S.A. The accession numbers listed in this specification are herein incorporated by reference as provided in the database on Sep. 17, 2008.

Administration: Providing or giving a subject an agent, such as a composition that includes a targeted cargo protein. Exemplary routes of administration include, but are not limited to, oral, injection (such as subcutaneous, intramuscular, intradermal, intraperitoneal, intratumoral and intravenous), sublingual, rectal or transrectal, transdermal, intranasal, vaginal, cervical, and inhalation routes. In specific examples, intratumoral includes local, regional, focal, or convection enhanced delivery. In other specific examples, administration includes transurethral or transperineal administration. In one example, surrogate magnetic resonance imaging tracers (e.g., gadolinium-bound albumin (Gd-albumin)) can be administered in combination with the targeted cargo protein to determine if the targeted cargo protein is delivered to a tumor, such as a brain tumor, safely at therapeutic doses while monitoring its distribution in real-time (see for example, Murad et al., Clin. Cancer Res. 12(10):3145-51 2006).

Antibody: Immunoglobulin molecules and immunologically active portions of immunoglobulin molecules, that is, molecules that contain an antigen binding site that specifically binds (immunoreacts with) an epitope, such as an epitope displayed by cancer stem cells. Antibodies include monoclonal antibodies, polyclonal antibodies, as well as humanized antibodies. Antibodies also include affibodies. Affibodies mimic monoclonal antibodies in function but are based on Protein A. Affibodies can be engineered as high-affinity ligands for binding to a targeting moiety.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CH1 domains; (ii) an Fd fragment consisting of the VH and CH1 domains; (iii) an Fv fragment consisting of the VL and VH domains of a single arm of an antibody (scFv) and scFv molecules linked to each other to form a bivalent dimer (diabody) or trivalent trimer (triabody); (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Methods of producing polyclonal and monoclonal antibodies are known to those of ordinary skill in the art, and many antibodies are available. See, e.g., Coligan, Current Protocols in Immunology Wiley/Greene, N.Y., 1991; and Harlow and Lane, Antibodies: A Laboratory Manual Cold Spring Harbor Press, NY, 1989; Stites et al., (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein, Nature 256: 495-497, 1975. Other suitable techniques for antibody preparation include selection of libraries of recombinant antibodies in phage or similar vectors. See, Huse et al., Science 246: 1275-1281, 1989; and Ward et al., Nature 341: 544-546, 1989.

Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984). Detailed methods for preparation of chimeric (humanized) antibodies can be found in U.S. Pat. No. 5,482,856. Additional details on humanization and other antibody production and engineering techniques can be found in Borrebaeck (ed), Antibody Engineering, 2nd Edition Freeman and Company, NY, 1995; McCafferty et al., Antibody Engineering, A Practical Approach, IRL at Oxford Press, Oxford, England, 1996, and Paul Antibody Engineering Protocols Humana Press, Towata, N.J., 1995.

In some examples, an antibody specifically binds to a target protein (e.g., a cell surface receptor such as an IL4 receptor) with a binding constant that is at least 10³ M⁻¹ greater, 10⁴ M⁻¹ greater or 10⁵ M⁻¹ greater than a binding constant for other molecules in a sample. In some examples, a specific binding reagent (such as an antibody (e.g., monoclonal antibody) or fragments thereof) has an equilibrium constant (K_{d}) of 1 nM or less. For example, a specific binding agent may bind to a target protein with a binding affinity of at least about 0.1×10⁻⁸ M, at least about 0.3×10⁻⁸M, at least about 0.5×10⁻⁸M, at least about 0.75×10⁻⁸ M, at least about 1.0×10⁻⁸ M, at least about 1.3×10⁻⁸ M at least about 1.5×10⁻⁸M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, N.J.

Binds or binding: The association between two or more molecules, wherein the two or more molecules are in close physical proximity to each other, such as the formation of a complex. An exemplary complex is a receptor-ligand pair or an antibody-antigen pair. Generally, the stronger the binding of the molecules in a complex, the slower their rate of dissociation. Specific binding refers to a preferential binding between an agent and a specific target. For example, specific binding refers to when a targeted cargo protein that includes a targeting moiety specific for a cancer stem cell antigen binds to the cancer stem cell, but does not significantly bind to other cells that do not display the target in close proximity to the cancer stem cell. Such binding can be a specific non-covalent molecular interaction between the ligand and the receptor. In a particular example, binding is assessed by detecting cancer stem cell growth inhibition using one of the methods described herein after the targeted cargo protein has been contacted with the cancer stem cell.

Such interaction is mediated by one or, typically, more noncovalent bonds between the binding partners (or, often, between a specific region or portion of each binding partner). In contrast to non-specific binding sites, specific binding sites are saturable. Accordingly, one exemplary way to characterize specific binding is by a specific binding curve. A specific binding curve shows, for example, the amount of one binding partner (the first binding partner) bound to a fixed amount of the other binding partner as a function of the first binding partner concentration. As the first binding partner concentration increases under these conditions, the amount of the first binding partner bound will saturate. In another contrast to non-specific binding sites, specific binding partners involved in a direct association with each other (e.g., a protein-protein interaction) can be competitively removed (or displaced) from such association (e.g., protein complex) by excess amounts of either specific binding partner. Such competition assays (or displacement assays) are very well known in the art.

Cancer: Malignant neoplasm that has undergone characteristic anaplasia with loss of differentiation, increased rate of growth, invasion of surrounding tissue, and is capable of metastasis. Residual cancer is cancer that remains in a subject after any form of treatment given to the subject to reduce or eradicate a cancer and recurrent cancer is cancer that recurs after such treatment. Metastatic cancer is a cancer at one or more sites in the body other than the site of origin of the original (primary) cancer from which the metastatic cancer is derived. In the case of a metastatic cancer originating from a solid tumor, one or more (for example, many) additional tumor masses can be present at sites near or distant to the site of the original tumor. The phrase “disseminated metastatic nodules” or “disseminated metastatic tumors” refers to a plurality (typically many) metastatic tumors dispersed to one or more anatomical sites. For example, disseminated metastatic nodules within the peritoneum (that is a disseminated intraperitoneal cancer) can arise from a tumor of an organ residing within or outside the peritoneum, and can be localized to numerous sites within the peritoneum. Such metastatic tumors can themselves be discretely localized to the surface of an organ, or can invade the underlying tissue.

Cargo Moiety: A peptide (e.g., protein fragment or full length protein) or other molecule that can function to significantly reduce or inhibit the growth of a cancer stem cell. In some examples a cargo moiety can trigger cell death (e.g., apoptosis). Exemplary cargo moieties include toxins, such as toxins derived from plants, microorganisms, and animals. In other examples, cargo moieties are proteins that normally contribute to the control of cell life cycles, for example a cargo moieties can be any protein that triggers cell death, such as via apoptotic or non-apoptotic pathways. In some examples, the cargo moiety is not a protein, but another molecule that can function to significantly reduce or inhibit the growth of a cancer stem cell, such as thapsigargin. In some examples, a cargo moiety is activated by a tumor-associated protease, such as PSA. Exemplary cargo moieties, and exemplary GenBank accession numbers, are provided in Table 1, below. In addition to native cargo sequences, variant sequences can also be used, such as mutant sequences with greater biological activity than that of the native sequence.

TABLE 1
Exemplary cargo moiety sequences

| Cargo Moiety | Accession Numbers* |
|---|---|
| Aerolysin | ABR14715.1; ABR14714.1 |
| Proaerolysin | AAA21938.1; P09167.2; U.S. Pat. No. 7,282,476 |
| | (proaerolysin sequences therein herein |
| | incorporated by reference) |
| Bouganin | AAL35962 and SEQ ID NO: 9 in U.S. Pat. No. |
| | 6,737,511, as well as variant sequences |
| | provided in U.S. Pat. No. 7,339,031 and WO |
| | 2005/090579 (bouganin sequences therein |
| | herein incorporated by reference) |
| Pseudomonas | 1IKP A; AAB59097.1; AAF90003.1 (also see |
| exotoxin | SEQ ID NO: 1 of U.S. Pat. No. 6,011,002) |
| Bcl-2 pro-apoptotic | BAD: CAG46757; AAH01901.1; CAG46733.1; |
| proteins such as | and sequences provided in U.S. Pat. No. 6,737,511 |
| BAD and BAX | BAX: CAE52909.1; AAO22992.1; EAW52418.1 |
| Cholera toxin | BAA06291.1; ACF35010.1; BAA06288.1; as well |
| | as variant sequences provided in U.S. patent |
| | application Ser. No. 61/058,872 (variant |
| | cholera toxin sequences therein herein |
| | incorporated by reference) |
| Ribonuclease A | BAA05124.1; NP_937877.1; NP_115961.2; |
| | Q5GAN4.1; and sequences provided in PCT |
| | Publication No. WO2007/041361 (rapLR1 |
| | sequences therein herein incorporated by |
| | reference) |

*GenBank Numbers are herein incorporated by reference, as well as their corresponding nucleic acid sequences.

Contact or contacting: Refers to the relatively close physical proximity of one object to another object. Generally, contacting involves placing two or more objects in close physical proximity to each other to give the objects and opportunity to interact. For example, contacting a targeted cargo protein with a cancer stem cell can be accomplished by placing the targeted cargo protein (which can be in a solution) in proximity to the cell, for example by injecting the targeted cargo protein into a subject having the cancer. Similarly, a targeted cargo protein can be contacted with a cell in vitro, for example by adding the targeted cargo protein to culture media in which the cell is growing.

Decrease: To reduce the quality, amount, or strength of something. In one example, a therapy (such as treatment with a targeted cargo protein) decreases a cancer stem cell population (such as by decreasing the size of a tumor, the volume of a tumor, the metastasis of a tumor, the number of cancer stem cells, or combinations thereof), or one or more symptoms associated with cancer, for example as compared to the response in the absence of the therapy. In a particular example, a therapy decreases the size of a tumor, volume of a tumor, number of cancer stem cells, or the metastasis of a cancer, or combinations thereof, subsequent to the therapy, such as a decrease of at least about 10%, at least about 20%, at least about 50%, or even at least about 90%. Such decreases can be measured using the methods disclosed herein.

Diagnose: The process of identifying a medical condition or disease, for example from the results of one or more diagnostic procedures. In particular examples, includes determining the prognosis of a subject (e.g., likelihood of survival over a period of time, such as likelihood of survival in 6-months, 1-year, or 5-years). In a specific example, cancer is diagnosed by detecting the presence of a cancer stem cell in a sample using one or more of the targets on the cancer stem cell surface. For example, diagnoses can include determining the particular stage of cancer or the presence of a site of metastasis.

Linker: A molecule used to connect one or more agents to one or more other agents. For example, a linker can be used to connect one or more cargo moieties to one or more targeting moieties. Particular non-limiting examples of linkers include dendrimers, such as synthetic polymers, peptides, proteins and carbohydrates. Linkers additionally can contain one or more protease cleavage sites or be sensitive to cleavage via oxidation and/or reduction.

Pharmaceutically acceptable carriers: The term “pharmaceutically acceptable carriers” refers to pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic or diagnostic agents, such as one or more of the targeted cargo protein molecules provided herein.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

Pharmaceutical agent or drug: A chemical compound or composition capable of inducing a desired therapeutic effect when administered to a subject, alone or in combination with another therapeutic agent(s) or pharmaceutically acceptable carriers. In a particular example, a pharmaceutical agent (such as one that includes a targeted cargo protein) treats a cancer, for example by reducing the size of the tumor (such as the volume or reducing the number of cancer stem cells), reducing metastasis of the cancer, or combinations thereof.

Recombinant: A recombinant molecule (such as a recombinant nucleic acid molecule or protein) has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. A recombinant protein is one that results from expressing a recombinant nucleic acid encoding the protein. Targeted cargo proteins of the present disclosure are generally recombinant.

Sample: Biological specimens such as samples containing biomolecules, such as nucleic acid molecules, proteins, or both. Exemplary samples are those containing cells or cell lysates from a subject, such as those present in peripheral blood (or a fraction thereof such as serum), urine, saliva, tissue biopsy, cheek swabs, surgical specimen, fine needle aspirates, cervical samples, and autopsy material. In a specific example, a sample is obtained from a tumor (for example a section of tissue from a biopsy), which can include tumor cells that are both non-cancer stem cells and cancer stem cells.

Sequence identity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN can be used to compare nucleic acid sequences, while BLASTP can be used to compare amino acid sequences. To compare two nucleic acid sequences, the options can be set as follows: —i is set to a file containing the first nucleic acid sequence to be compared (such as C:\seq1.txt); —j is set to a file containing the second nucleic acid sequence to be compared (such as C:\seq2.txt); —p is set to blastn; —o is set to any desired file name (such as C:\output.txt); —q is set to −1; —r is set to 2; and all other options are left at their default setting. For example, the following command can be used to generate an output file containing a comparison between two sequences: C:\Bl2seq—i c:\seq1.txt—j c:\seq2.txt—p blastn—o c:\output.txt—−1—r 2.

To compare two amino acid sequences, the options of Bl2seq can be set as follows: —i is set to a file containing the first amino acid sequence to be compared (such as C:\seq1.txt); —j is set to a file containing the second amino acid sequence to be compared (such as C:\seq2.txt); —p is set to blastp; —o is set to any desired file name (such as C:\output.txt); and all other options are left at their default setting. For example, the following command can be used to generate an output file containing a comparison between two amino acid sequences: C:\Bl2seq—i c:\seq1.txt—j c:\seq2.txt—p blastp—o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a cargo protein or targeting moiety provided herein.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity to a cargo moiety or targeting moiety provided herein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

Subject: Living multi-cellular vertebrate organisms, a category that includes human and non-human mammals (such as laboratory or veterinary subjects).

Targeted Cargo Protein: Any protein that binds specifically to a cancer stem cell and reduces or inhibits cancer stem cell growth, or kills cancer stem cells. In some examples, targeted cargo proteins can target both cancer stem cells and tumor (e.g., cancer) cells that are not cancer stem cells. Targeted cargo proteins include a targeting moiety and a cargo moiety, the targeting moiety specifically binds with the cancer stem cell and the cargo moiety significantly reduces or inhibits the growth of the cancer stem cell or kills cancer stem cells. In some examples the cargo moiety causes the death of the cancer stem cell that it is associated with. Because in some examples the cargo moiety is not a protein, such as a chemotherapeutic agent, and in some examples the targeting moiety is not a protein, the targeted cargo protein in some examples is not actually a protein.

Targeting moiety: Any compound that binds to a molecule (herein referred to as a target) displayed by a cancer stem cell, for example a targeting moiety can be an antibody that binds to a target (e.g., receptor), a ligand (e.g., a cytokine or growth factor) that binds to a receptor, a permuted ligand that binds to a receptor, or a peptide sequence sensitive to cleavage by a tumor-associated protease. In some examples, a targeting moiety is activated by a tumor-associated protease, such as PSA. Typically, targeting moieties selectively bind to one type of cell displaying a target more effectively than they bind to other types of cells that do not display the target. Targeting moieties can be chosen to selectively bind to subsets of tumor cells, such as cancer stem cells. Targeting moieties include specific binding agents such as antibodies, natural ligands of the target on the stem cell, such as IL-4, derivatives of such natural ligands, and immunoglobulin A. In some examples, the targeting moiety is not biologically active (e.g., cannot activate a receptor), but retains the ability to bind to the target and thus direct the targeted cargo protein to the appropriate cells. Other exemplary targeting moieties include the protein (not yet fully characterized) which binds to the 8H9 monoclonal antibody (see WO 2004/050849).

Table 2 provides information relating to the sequences of exemplary natural ligands as well as other antigens that can be used as targeting moieties. In some examples, circular permuted ligands, such as circular permuted IL-4, can be used to bind cancer stem cells. As additional research is performed, new cancer stem cell specific targets will be identified. These additional markers can be used as targets for binding to targeting moieties and targeted cargo proteins can be made to inhibit the growth of (or kill) cancer stem cells displaying such ligands. One of ordinary skill in the art will appreciate that once a marker is known, standard methods of making antibodies to the identified marker can be used to make targeting moieties specific for the cancer stem cell marker, thus, allowing for the development of a specific targeted cargo protein.

TABLE 2
Exemplary targeting moiety sequences

| Receptor or Antigen | |
|---|---|
| to be Targeted | Accession Number* |
| EGF | NP_001954; EAX06257.1; AAR84237.1 |
| EpCAM | NP_002345; NP_032558.2; NP_612550.1 |
| IL-2 | CAA07317; AAB46883.1; NP_000577.2 |
| IL-3 | AAC08706.1; AAA99502.1; CAE45598.1 |
| IL-4 | AAH70123; CAA57444.1; AAH67515.1 (also see |
| | SEQ ID NO: 2 and various circularly permuted |
| | ligands in U.S. Pat. No. 6,011,002) |
| IL-5 | NP_000870.1; CAA01794.1; P32927.2 |
| IL-13 | AAH96141.2; AAH96138.1; AAH96139.1 |
| GMCSF | P04141.1; AAI13925.1; AAI08725.1 |
| Tenascin | AAA36728.1; CAA39628.1; NP_002151.2 |
| Mesothelin | CAC37289.1; ABW03459.1; AAH09272.1; |
| | AAH03512.1; as well as the mesothelins |
| | disclosed in U.S. Pat. Nos. 7,081,518 |
| | and 6,051,405 (mesothelin sequences therein |
| | herein incorporated by reference) |
| CD22 | BAA36575.1; BAA36576.1; BAA36567.1 |
| PSMA (also known as | ABO93402.2; AAC83972.1; NP_001014986.1; |
| folate hydrolase) | NP_004467.1 |

*GenBank Numbers are herein incorporated by reference, as well as their corresponding nucleic acid sequences.

Targets on cancer stem cells include small molecules displayed on the surface of cancer stem cells. Antibodies directed to such targets can be used as targeting moieties as well as the natural ligands of the targets and derivatives thereof.

Therapeutically effective amount: An amount of an agent that alone, or together with a pharmaceutically acceptable carrier or one or more additional therapeutic agents, induces the desired response. A therapeutic agent, such as a targeted cargo protein, is administered in therapeutically effective amounts that stimulate the desired response, for example reduction of symptoms of cancer in subjects known to have a cancer that includes cancer stem cells.

Effective amounts of a therapeutic agent can be determined in many different ways, such as assaying for improvement of a physiological condition of a subject having cancer. Effective amounts also can be determined through various in vitro, in vivo or in situ assays.

Therapeutic agents can be administered in a single dose, or in several doses, for example weekly, monthly, or bi-monthly, during a course of treatment. However, the effective amount of can be dependent on the source applied, the subject being treated, the severity and type of the condition being treated, and the manner of administration.

In one example, it is an amount sufficient to partially or completely alleviate symptoms of cancer in a subject. Treatment can involve only slowing the progression of the cancer temporarily, but can also include halting or reversing the progression of the cancer permanently. For example, a pharmaceutical preparation can decrease one or more symptoms of the cancer (such as the size of a tumor or the number of tumors or number of cancer stem cells), for example decrease a symptom by at least about 20%, at least about 50%, at least about 70%, at least about 90%, at least about 98%, or even at least about 100%, as compared to an amount in the absence of the therapeutic preparation.

Treating a disease: A therapeutic intervention that ameliorates a sign or symptom of a disease or pathological condition, such a sign or symptom of cancer. Treatment can also induce remission or cure of a condition, such as cancer. In particular examples, treatment includes preventing a disease, for example by inhibiting the full development of a disease, such as preventing development of tumor metastasis. Prevention of a disease does not require a total absence of a dysplasia or cancer. For example, a decrease of at least about 50% can be sufficient.

Tumor: Is a neoplasm or an abnormal mass of tissue that is not inflammatory, which arises from cells of preexistent tissue. A tumor can be either benign (noncancerous) or malignant (cancerous). Tumors can be solid or hematological. Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelogenous leukemia, and chronic lymphocytic leukemia), myelodysplastic syndrome, and myelodysplasia, polycythemia vera, lymphoma, (such as Hodgkin's disease, all forms of non-Hodgkin's lymphoma), multiple myeloma, Waldenstrom's macroglobulinemia, and heavy chain disease.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, lung cancer, ovarian cancer, prostate cancer, benign prostatic hyperplasia, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, epithelial tumors (e.g., cervical cancer, gastric cancer, skin cancer, head and neck tumors), testicular tumor, bladder carcinoma, melanoma, brain tumors, and CNS tumors (such as a glioma, astrocytoma, medulloblastoma, craniopharyogioma, ependymoma, pinealoma, hemangioblastoma, acoustic neuroma, oligodendroglioma, menangioma, meningioma, neuroblastoma and retinoblastoma).

Under conditions sufficient for: A phrase that is used to describe any environment that permits the desired activity. In one example, includes incubating a targeted cargo protein with tumor stem cell under conditions that allow the targeted cargo protein to specifically bind to a cancer stem cell in the sample. In another example, includes contacting one or more targeted cargo proteins with one or more cancer stem cells in a subject sufficient to allow the desired activity. In particular examples, the desired activity is decreasing growth or multiplication of such cancer stem cells or killing cancer stem cells.

Unit dose: A physically discrete unit containing a predetermined quantity of an active material (such a targeted cargo protein) calculated to individually or collectively produce a desired effect such as a therapeutic effect. A single unit dose or a plurality of unit doses can be used to provide the desired effect, such as a therapeutic effect.

### II. Overview of Several Embodiments

Described herein are proteins or other agents that target cancer stem cells and inhibit growth of and/or kill cancer stem cells. The molecules, herein after collectively referred to as targeted cargo proteins, include a targeting moiety that binds to a target displayed by the cancer stem cell as well as a cargo moiety that provides the cell growth inhibiting (or cell killing) activity. The targeting moiety can be bound to the cargo moiety directly or through one or more of a variety of linkers that are further described herein. Cancer stem cells generally have the ability to self-renew and thus generate progeny with similar properties as themselves. In some examples, the disclosed targeted cargo proteins can target both cancer stem cells and tumor (e.g., cancer) cells that are not cancer stem cells. Therefore, in some examples targeted cargo proteins can kill or inhibit the growth of cancer stem cells and tumor (e.g., cancer) cells that are not cancer stem cells. In other examples, such as with a targeting moiety directed to CD 133, the targeted cargo proteins kill or inhibit the growth of cancer stem cells in the tumor, but not tumor cells that are not cancer stem cells.

Targeting moieties include proteins and other agents that function to specifically bind to a target on a cancer stem cell (but in some examples the target may also be present on other cancer cells). Targeting moieties include specific binding agents, such as antibodies, affibodies, or receptor ligands. In some examples, the targeting moiety is derived from the natural ligand to the target (e.g., cell surface receptor) displayed by the cancer stem cell. The targeting moiety that is derived from a natural ligand can include the complete amino acid sequence of the ligand (e.g. the same sequence that the ligand would have if it was isolated from nature), or the amino acid sequence of the targeting moiety can share at least about 95%, at least about 90%, at least about 80%, at least about 70%, at least about 60%, at least about 50%, or at least about 40% sequence identity with the natural ligand (e.g., at least about this amount of sequence identity to the GenBank Accession Nos. listed in Table 2), as long as the variant retains or has enhanced biological activity of the native ligand. In some examples, such variants have an increased binding affinity for their target relative to the native ligand. A targeting moiety that is derived from a natural ligand can also be a fragment of the native sequence that is capable of binding to the target displayed by the cancer stem cell. In some examples, the ligand is a circularly permuted version of a natural ligand (e.g., see U.S. Pat. No. 6,011,002). Circularly permuted molecules include those in which the termini of a linear molecule (e.g., ligand) have been joined together, either directly or via a linker, to produce a circular molecule, and then the circular molecule is opened at another location to produce a new linear molecule with termini different from the termini in the original molecule. In some examples, the targeting moiety has one or more amino acid mutations (relative to the native sequence), which alters binding to the target, such as a mutations that increase binding of a ligand to its target.

Cargo moieties can reduce, inhibit the growth of, and/or kill cancer stem cells, and in some examples also inhibit the growth of, and/or kill bulk cancer cells (e.g., non stem cancer cells). These molecules can be native proteins, or proteins that have been engineered, as well as other molecules that inhibit the growth of, and/or kill cancer stem cells, and in some examples also inhibit the growth of, and/or kill bulk cancer cells (e.g., non stem cancer cells). One example of such a molecule is a chemotherapeutic agent, such as thapsigargin. Cargo moieties can be linked to targeting moieties (a linked cargo moiety and targeting moiety is referred to herein as a targeted cargo protein) that bind to cancer stem cells. Thus, the cargo moiety linked to the targeting moiety will bind to the cancer stem cell and inhibit the growth of (or kill) the cancer stem cell. In some examples, the cargo moiety can cause cancer stem cell death and in some examples the cancer stem cell death is caused by apoptosis. In some examples cargo moieties are toxins (including plant or microorganism derived toxins), active fragments of toxins, or derivatives of toxins that share at least about 95%, at least about 90%, at least about 80%, at least about 70%, at least about 60%, at least about 50%, or at least about 40% sequence identity with the natural toxin and retains or has enhanced biological activity of the native toxin, for example with the cargo moieties provided in Table 1. In other examples the cargo moieties are derived from proteins that modulate cell life cycles or are part of natural immune responses in animals. For example, some cargo moieties are derived from proteins that are known to induce apoptosis. In some examples cargo moieties are derived from pro-apoptotic proteins, active fragments of such proteins, or derivatives of such proteins that share at least about 95%, at least about 90%, at least about 80%, at least about 70%, at least about 60%, at least about 50%, or at least about 40% sequence identity with the natural moiety (see Table 1 for sequence accession numbers), as long as the variant retains or has enhanced biological activity of the native moiety. In additional examples a cargo moiety can be inactive when administered as part of a targeted cargo protein, and then upon contacting another molecule in the subject become active. A more detailed description of cargo moieties is provided herein.

The description also includes methods of treating subjects having (or had) cancer with the targeted cargo protein. For example, the method can include administering one or more disclosed targeted cargo proteins to the subject, thereby treating cancer stem cells in the subject (e.g., reducing the number or volume of stem cells). For example, the targeted cargo proteins can be used to treat subjects with recurrent cancer or cancer that is refractory. In such examples the subject is treated with a traditional anti-cancer therapy, for example radiation, surgery, or chemotherapy and then tested to determine the effectiveness of the treatment. If the traditional therapy did not alter the cancer in a desired way, the subject can then be treated with a targeted cargo protein.

In some examples treatment regimes that include targeted cargo proteins and additional anticancer therapeutics can be administered to a subject. The targeted cargo protein and the additional anticancer therapeutic will vary depending upon the type of cancer stem cell being targeted.

In specific examples, a subject is administered one or more of the following specific targeted cargo proteins to treat cancer stem cells: circularly permuted IL-4-Pseudomonas exotoxin (see U.S. Pat. No. 6,011,002), IL-2-aerolysin (see WO 2007/140618), IL-2-proaerolysin (see WO 2007/140618), EGF-proaerolysin, IL-4-BAD, anti-EpCAM-Pseudomonas exotoxin (EpCAM-PE), anti-EpCAM-bouganin, GMCSF-BAD, anti-mesothelin antibody-PE, anti-CD22 antibody-PE, anti-CD22 antibody-RNase A (rapLR1) and anti-PSMA antibody-thapsigargin or other chemotherapeutic agents.

### III. Targeted Cargo Proteins

Targeted cargo proteins are proteins that include a targeting moiety linked to a cargo moiety. Targeted cargo proteins function to specifically bind to cancer stem cells and reduce or inhibit cancer stem cell growth.

A. Cargo Moieties

Cargo moieties reduce or inhibit cancer stem cell growth, or kill cancer stem cells. In some examples cargo moieties are not proteins, but other molecules that reduce or inhibit cancer stem cell growth, or kill cancer stem cells, such as chemotherapeutic agents. In some examples, cargo moieties also reduce or inhibit bulk cancer cell growth, or kill cancer cells. Any protein or other agent that functions to reduce or inhibit cancer stem cell growth, or kill such cells, can be used as a cargo moiety. For example, toxins and proteins that function to control cell life cycles can be used as cargo moieties. Toxins that can be used as cargo moieties include toxins made by microorganisms, plants or animals, as well as toxins made by human cells. Similarly, any natural cell growth controlling protein can be used as a cargo moiety. For example, proteins that trigger cell death during the normal life cycle of an organism can be used as cargo moieties. In some examples, an oncolytic virus (e.g., see Allen et al., Mol. Ther. 16:1556-64, 2008) or liposomes carrying cytotoxic agents (e.g., see Madhankumar et al., Mol. Cancer. Ther. 5:3162-9, 2006) is used as the cargo protein.

In one example, the cargo moiety is a toxin. Exemplary toxins that can be used include pore-forming toxins, and toxins that upon internalization inhibit cell growth. In other examples, cargo moieties are proteins that are apoptotic triggering proteins, and cell growth inhibiting proteins. In some examples, the toxin is a modified bacterial toxin such that the resulting toxin is less immunogenic than the native toxin. Such modified toxins, such as a modified Pseudomonas exotoxin A, can reduce the patient's immunogenic response, thereby allowing repeated administration.

Pore forming toxins are toxins that form pores in the cell membrane thereby killing the cell via cell lyses. Exemplary pore forming toxins include but are not limited to human toxins such as perforin or bacterial toxins such as aerolysin as well as modified pore-forming protein toxins that are derived from naturally occurring pore-forming protein toxins (nPPTs) such as aerolysin or aerolysin-related polypeptides. Suitable aerolysin-related nPPTs have the following features: a pore-forming activity that is activated by removal of an inhibitory domain via protease cleavage, and the ability to bind to receptors that are present on cell membranes through one or more binding domains. In some examples the linker can be engineered to be sensitive to a protease or be chemically liable. Additional examples of pore forming toxins that can be used as cargo moieties include, but are not limited to, proaerolysin from Aeromonas hydrophila, Aeromonas trota and Aeromonas salmonicida, alpha toxin from Clostridium septicum, anthrax protective antigen, Vibrio cholerae VCC toxin, epsilon toxin from Clostridium perfringens, and Bacillus thuringiensis delta toxins. A detailed description of the engineering of proaerolysin can be found in U.S. Pat. No. 7,282,476, which is herein incorporated by reference.

Additional toxins that can be used as cargo moieties include toxins that act within a cell. For example, anthrax, diphtheria, cholera, and botulinum toxins include a portion that acts in the cytoplasm, as well as a portion that acts to bind to the cell surface. These toxins, or portions thereof, can be linked to a targeting moiety and used to inhibit cancer stem cell growth. Select members of the ribonuclease A (RNase A) superfamily are potent cytotoxins. These cytotoxic ribonucleases enter the cytosol, where they degrade cellular RNA and cause cell death.

In some examples ribosome inactivating proteins can be used as toxins. In these examples the cargo moiety is a polypeptide having ribosome-inactivating activity including, without limitation, gelonin, bouganin, saporin, ricin, ricin A chain, bryodin, restrictocin, and variants thereof. Diphtheria toxin and Pseudomonas exotoxin A inhibit protein synthesis via ADP-ribosylation of elongation factor 2. When the cargo moiety is a ribosome-inactivating protein or inhibits protein synthesis via ADP-ribosylation of elongation factor 2, the targeted cargo protein can be internalized upon binding to the cancer stem cell.

Cargo moieties that induce apoptosis can also be used to target cancer stem cells. Examples of cargo moieties that induce apoptosis include caspases, granzymes and BCL-2 pro-apoptotic related proteins such as BAX (e.g., Accession no: CAE52910), BAD (e.g., Accession no: CAG46757), BAT (e.g., Accession no: AAI07425), BAK (e.g., Accession no: AAA74466), BIK (e.g., Accession no: CAG30276), BOK (e.g., Accession no: AAH06203), BID (e.g., Accession no: CAG28531), BIM (e.g., Accession no: NP_{—}619527) and BMF (e.g., Accession no: AAH69328). These cargo moieties can be used alone of in combination to reduce or inhibit cancer stem cell growth.

Aerolysin is a channel-forming toxin produced as an inactive protoxin called proaerolysin (PA). Exemplary aerolysin and PA sequences that can be used in a targeted cargo protein are provided in Table 1. The PA protein contains many discrete functionalities that include a binding domain, a toxin domain, and a C-terminal inhibitory peptide domain that contains a protease activation site. The binding domain recognizes and binds to glycophosphatidylinositol (GPI) membrane anchors, such as are found in Thy-1 on T lymphocytes, the PIGA gene product found in erythrocyte membranes and Prostate Stem Cell Antigen (PSCA). The activation or proteolysis site within proaerolysin is a six amino acid sequence that is recognized as a proteolytic substrate by the furin family of proteases. PA is activated upon hydrolysis of a C-terminal inhibitory segment by furin. Activated aerolysin binds to GPI-anchored proteins in the cell membrane and forms a heptamer that inserts into the membrane producing well-defined channels of ˜17 Å. Channel formation leads to rapid cell death. Wild-type aerolysin is toxic to mammalian cells, including erythrocytes, for example at 1 nanomolar or less.

In some examples, a target cargo protein is an PA molecule with the native furin site replaced with a different cleavage site, such as prostate-specific protease cleavage site (e.g., a PSA-specific cleavage site, which permits activation of the variant PA in the presence of a prostate-specific protease such as PSA, PMSA, or HK2). In one example, a prostate-specific protease cleavage site is inserted into the native furin cleavage site of PA, such that PA is activated in the presence of a prostate-specific protease, but not furin. In another example, a variant PA molecule further includes a functionally deleted binding domain (e.g., about amino acids 1-83 of a native PA protein sequence). Functional deletions can be made using any method known in the art, such as deletions, insertions, mutations, or substitutions. In some examples, targeted cargo proteins include variant PA molecules in which the native binding domain is functionally deleted and replaced with a prostate-tissue or other tissue-specific binding domain. In other examples, variant PA molecules include a furin cleavage site and a functionally deleted binding domain which is replaced with a prostate-tissue specific binding domain. Such variant PA molecules are targeted to prostate cells via the prostate-tissue specific binding domain, and activated in the presence of furin.

Bouganin is a ribosome-binding protein originally isolated from Bougainvillea speotabilis (see U.S. Pat. No. 6,680,296). Exemplary modified bouganins are described in WO 2005/090579 and U.S. Pat. No. 7,339,031. Bouganin damages ribosomes and leads to a cessation of protein synthesis and cell death. Exemplary bouganin proteins that can be used in the targeted cargo proteins of the present disclosure include those in GenBank Accession No. AAL35962, as well as those native and modified bouganin sequences provided in U.S. Pat. Nos. 6,680,296; 7,339,031 and PCT publication WO 2005/090579 (bouganin sequences herein incorporated by reference), as well as sequences having at least 60% sequence identity, at least 75%, at least 80%, at least 85%, at least 90%, at least 95%, at least 98% or even at least 99% sequence identity to such sequences.

BAD, BCL2-associated agonist of cell death, is a regulator of programmed cell death (apoptosis). BAD positively regulates cell apoptosis by forming heterodimers with BCL-xL and BCL-2, and reversing their death repressor activity. Proapoptotic activity of BAD is regulated through its phosphorylation. Exemplary BAD proteins that can be used in the targeted cargo proteins of the present disclosure include those in GenBank Accession Nos. CAG46757; AAH01901.1; and CAG46733.1, as well as those sequences provided in U.S. Pat. No. 6,737,511 (sequences herein incorporated by reference), as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native BAD protein.

BAX, BCL2-associated X protein, is a regulator of programmed cell death (apoptosis). This protein forms a heterodimer with BCL2, and functions as an apoptotic activator. BAX interacts with, and increases the opening of, the mitochondrial voltage-dependent anion channel (VDAC), which leads to the loss in membrane potential and the release of cytochrome c. Exemplary BAX proteins that can be used in the targeted cargo proteins of the present disclosure include those provided by GenBank Accession Nos. CAE52909.1; AA022992.1; EAW52418.1, U.S. Pat. No. 6,645,490 (Bax in the IL2-Bax construct is a Bax-alpha variant that can be used in the present disclosure), as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native BAX protein.

In some examples, the BAX protein of a targeted cargo protein may be modified such that the C-terminal anchor domain has been deleted and replaced with a CaaX sequence. CaaX is a peptide with the sequence Cysteine-a-a-X where “X” is any amino acid and “a” is an aliphatic amino acid. Because membrane association of BAX is needed for optimal apoptosis activity, addition of membrane binding domains such as CaaX can enhance their pro-apoptotic activities. Proteins with CaaX sequence are farnesylated. Farnesylated proteins are targeted to membranes (e.g., see Wright and Philip, J. Lipid Res., 2006, 47(5): 883-91). Potential BAX variants containing a CaaX sequence may or may not contain the C-terminal anchor domain.

Pseudomonas exotoxin (PE) is a toxin secreted by Pseudomonas. Native PE is cytotoxic for mammalian cells due to its ability to enter cells by receptor-mediated endocytosis and then, after a series of intracellular processing steps, translocate to the cell cytosol and ADP-ribosylate elongation factor 2. This results in the inhibition of protein synthesis and cell death. PE has three functional domains: an amino-terminal receptor-binding domain, a middle translocation domain, and a carboxyl-terminal ADP-ribosylation domain. Modified PE molecules can include elimination of domain Ia, as well as deletions in domains II and III. Exemplary PE proteins that can be used in the targeted cargo proteins of the present disclosure include those provided in Table 1, as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native PE protein.

Thapsigargin is an inhibitor of sarco/endoplasmic reticulum Ca2+ ATPases. Thapsigargin is classified as a sesquiterpene lactone, and raises cytosolic calcium concentration by blocking the ability of the cell to pump calcium into the sarcoplasmic and endoplasmic reticulum which causes these stores to become depleted. Store-depletion can secondarily activate plasma membrane calcium channels, allowing an influx of calcium into the cytosol.

Ribonuclease A (RNAseA) is an endonuclease that cleaves single-stranded RNA. RNAse A toxins can be obtained from mammals and reptiles. Exemplary RNAse A proteins that can be used in the targeted cargo proteins of the present disclosure include those provided in Table 1, as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native RNAseA toxin.

The cargo moiety used can include native sequences (such as the GenBank Accession Nos. and sequences present in the patents referenced in Table 1 and listed above), as well as variants thereof, such as a variant having at least 98%, at least 95%, at least 90%, at least 80%, at least 70%, or at least 60% sequence identity with the native cargo moiety, as long as the variant retains or has enhanced biological activity of the native cargo moiety (e.g., at least about this amount of sequence identity to the GenBank Accession Nos. listed in Table 1 and listed above). In some examples, variant sequences retain substantially the same amount (or even more) of the native biological function of the cargo moiety, such as the ability to kill or inhibit the growth of a cancer stem cell. A cargo moiety can also be a fragment of the native sequence that retains a substantial amount of the native biological function of the protein.

The cargo moieties are engineered to target cancer stem cells by linking them to targeting moieties. Targeting moieties include agents that can bind to cancer stem cell surface targets.

B. Cancer Stem Cell Targeting Moieties

Targeting moieties are the portion of the targeted cargo proteins that target the targeted cargo protein to cancer stem cells, and in some example also bulk cancer cells. Targeting moieties function to specifically bind to a cancer stem cell. However, it is appreciated that the targeting moiety need not retain its native biological activity (e.g., the ability to activate a receptor or ability to prevent a ligand from binding to its receptor) as long as it permits the targeted cargo protein to bind with high specificity to cancer stem cells (and in some examples also cancer cells). In certain examples, the targeting moiety is a natural ligand of a target displayed by the cancer stem cell or a derivative of a natural ligand. In other examples the targeting moiety is an antibody, such as a humanized antibody or antibody fragment, which specifically binds to a target displayed on the surface of the cancer stem cell (e.g., targets a receptor). Targeting moieties can be linked to cargo moieties using any method known in the art, for example via chemical or recombinant technology.

A non-limiting list of compounds that could be used to target cancer stem cells includes antibodies, natural ligands, engineered ligands and combinations thereof that bind to one or more cancer stem cells. Exemplary ligands include cytokines and growth factors. Exemplary targets on cancer stem cells include IL-2R, IL-4R, IL-13R, IL-3R, IL-5R, GMCSFR, IL-10R, EGFR, transforming growth factor alpha (TGF-alpha), EpCAM, mesothelin, tenascin, CD22, CD30, PSMA, alpha PDGFR, human transmembrane glycoprotein NMB, antigen recognized by the 8H9 monoclonal antibody, cell surface markers such as CD133, CD132, CD124, CD117, CD90, CD71, CD45, CD44, CD38, CD34, CD24 and CD20 and cell surface receptors which may regulate downstream signaling pathways such as Notch, Hedgehog, Wnt and Bmi-1.

Of particular interest are targeting moieties that are molecules that are natural ligands or derivatives of the natural ligands to the target on the cancer stem cells. For example, if the cancer stem cell expresses IL-4 receptors (IL-4R), IL-4 ligand can be used as the targeting moiety. The IL-4 can be chemically or recombinantly linked to one or more of the cargo moieties described herein. Examples of derivatives of natural ligands include the circularized cytokine ligands described in U.S. Pat. No. 6,011,002 to Pastan et al., which is herein incorporated by reference. In addition to IL-4 ligands, IL-13 can also be used as a ligand targeting moiety since the IL-4 and IL-13 receptors share some sequence and biological functions.

Similarly, IL-2, EGF, and GMCSF can be used as targeting moieties to target cancer stem cells expressing the receptors for IL-2, EGF, and GMCSF, respectively. As described above, the targeting moiety can include the amino acid sequence of these ligands, as well as variants or fragments thereof (see Table 2 for exemplary accession numbers) that function to specifically bind the associated receptor. IL-3 and IL-5 can also be used as targeting moieties since they share a common receptor subunit with the GMCSF receptor.

In some examples, antibodies (including fragments, humanized antibodies and the like as described above) that target a receptor or other protein on a cancer stem cell are used as targeting moieties (e.g., specifically bind to receptors of IL-2, IL-4, EGF, or GMCSF or to EpCAM, PSMA, mesothelin, CD22, CD30, tenascin, NMB or 8F19 antigen). Antibodies are commercially available from various companies such as Millipore, Bedford, Mass. or custom made antibodies can be ordered from companies such as Cambridge Research Biochemicals, Billingham, Cleveland. Methods routine in the art can be used to generate such antibodies if desired. Such antibodies will specifically bind to cancer stem cells (and may also bind to bulk cancer cells) and function to place the cargo moiety in contact with a cancer stem cell.

IL-2 is a secreted cytokine involved in the proliferation of T and B lymphocytes. The IL-2 receptor is a heterotrimeric protein complex whose gamma chain is also shared by interleukin 4 (IL-4) and interleukin 7 (IL-7). Exemplary IL-2 proteins that can be used in the targeted cargo proteins of the present disclosure include those provided in Table 2, as well as sequences having at least 60% sequence identity, at least 75%, at least 80%, at least 85%, at least 90%, at least 95%, at least 98% or even at least 99% sequence identity to such sequences, as long as the variant retains the ability to bind the IL-2 receptor.

IL-4 is a pleiotropic cytokine produced by activated T cells, and is the ligand for the IL-4 receptor. The IL-4 receptor also binds to IL-13. Thus, IL-13 can also be used as a targeting moiety to target the IL-4 receptor. IL-4, IL-3, IL-5, IL-13, and CSF2 form a cytokine gene cluster on human chromosome 5q, with this gene particularly close to IL-13. Exemplary IL-4 and IL-13 proteins that can be used in the targeted cargo proteins of the present disclosure include those provided in Table 2, as well as sequences having at least 60% sequence identity, at least 75%, at least 80%, at least 85%, at least 90%, at least 95%, at least 98% or even at least 99% sequence identity to such sequences, as long as the variant retains the ability to bind the IL-4 receptor.

EGF is a growth factor that plays a role in the regulation of cell growth, proliferation, and differentiation by binding to its receptor EGFR. Human EGF is a 6045-Da protein with 53 amino acid residues and three intramolecular disulfide bonds. The EGF receptor is a member of the ErbB family of receptors. Exemplary EGF proteins that can be used in the targeted cargo proteins (e.g., to target EGFR on the surface of cancer stem cells) of the present disclosure include those provided in Table 2, as well as sequences having at least 60% sequence identity, at least 75%, at least 80%, at least 85%, at least 90%, at least 95%, at least 98% or even at least 99% sequence identity to such sequences, as long as the variant retains the ability to bind the EGF receptor.

EpCAM, also known as tumor-associated calcium signal transducer 1 (TACSTD-1), is encoded by a 9-exon gene on human chromosome 2, 2p21. EpCAM is a transmembrane glycoprotein expressed on epithelial cells, which is differentially expressed in most carcinomas and functions as a homotypic calcium-independent cell adhesion molecule. Exemplary EpCAM-target proteins that can be used in the targeted cargo proteins of the present disclosure include those provided in Table 2, as well as sequences having at least 60% sequence identity, at least 75%, at least 80%, at least 85%, at least 90%, at least 95%, at least 98% or even at least 99% sequence identity to such sequences. In some examples, EpCAM is targeted using a single-chain antibody fragment specific for the EpCAM antigen. In one example, the targeting moiety is an anti-EpCAM antibody (e.g., see U.S. Pat. No. 7,033,798).

GMCSF is a cytokine that functions as a white blood cell growth factor. GMCSF stimulates stem cells to produce granulocytes (neutrophils, eosinophils, and basophils) and monocytes. The GMCSF receptor is overexpressed in many leukemia and solid tumors. The GMCSF receptor includes both an alpha and beta subunit. The GMCSF receptor beta subunit also binds IL-3 and IL-5. Thus, IL-3 and IL-5 ligands, in addition to GMCSF, can be used to target GMCSF receptors on the surface of cancer stem cells. Exemplary GMCSF, IL-3 and IL-5 proteins that can be used in the targeted cargo proteins of the present disclosure include those provided in Table 2, as well as sequences having at least 60% sequence identity, at least 75%, at least 80%, at least 85%, at least 90%, at least 95%, at least 98% or even at least 99% sequence identity to such sequences, as long as the variant retains the ability to bind the GMCSF, IL-3 or IL-5 receptor.

Tenascin is a glycoprotein expressed on the cell surface of many cancer cells. There are four members of the tenascin gene family: tenascin-C, tenascin-R, tenascin-X and tenascin-W. Exemplary tenascin-targeting proteins that can be used in the targeted cargo proteins of the present disclosure include tenascin-C-specific antibodies, as well such as antibodies specific for the sequences provided in Table 2, including sequences having at least 60% sequence identity, at least 75%, at least 80%, at least 85%, at least 90%, at least 95%, at least 98% or even at least 99% sequence identity to such sequences.

Mesothelin (Uniprot Q13421) is a 40 kDa protein present on normal mesothelial cells and overexpressed in several human tumors, including mesothelioma and ovarian and pancreatic adenocarcinoma. The mesothelin gene encodes a precursor protein that is processed to yield mesothelin which is attached to the cell membrane by a glycosylphosphatidyl inositol linkage and a 31-kDa shed fragment named megakaryocyte-potentiating factor (MPF). Exemplary mesothelin-targeting proteins that can be used in the targeted cargo proteins of the present disclosure include those provided in Table 2, as well as sequences having at least 60% sequence identity, at least 75%, at least 80%, at least 85%, at least 90%, at least 95%, at least 98% or even at least 99% sequence identity to such sequences. In some examples, mesothelin is targeted using a single-chain antibody fragment specific for the mesothelin antigen. In one example, the targeting moiety is an anti-mesothelin antibody.

CD22 (cluster of differentiation-22) is a regulatory molecule that prevents the overactivation of the immune system and the development of autoimmune diseases. Exemplary CD22-target proteins that can be used in the targeted cargo proteins of the present disclosure include those provided in Table 2, as well as sequences having at least 60% sequence identity, at least 75%, at least 80%, at least 85%, at least 90%, at least 95%, at least 98% or even at least 99% sequence identity to such sequences, as long as the variant retains the ability to bind to the CD22 receptor. In some examples, CD22 is targeted using a single-chain antibody fragment specific for the CD22 antigen. In one example, the targeting moiety is an anti-CD22 antibody.

PSMA (prostate specific membrane antigen), also known as folate hydrolase, is a type II transmembrane glycoprotein belonging to the M28 peptidase family. The protein acts as a glutamate carboxypeptidase on different alternative substrates, including the nutrient folate and the neuropeptide N-acetyl-l-aspartyl-l-glutamate and is expressed in a number of tissues such as prostate, central and peripheral nervous system and kidney. Exemplary PSMA-targeting proteins that can be used in the targeted cargo proteins of the present disclosure include those provided in Table 2, as well as sequences having at least 60% sequence identity, at least 75%, at least 80%, at least 85%, at least 90%, at least 95%, at least 98% or even at least 99% sequence identity to such sequences. In some examples, PSMA is targeted using a single-chain antibody fragment specific for the PSMA antigen. In one example, the targeting moiety is an anti-PSMA antibody.

The targeting moiety used can include native sequences (such as the GenBank Accession Nos. and sequences present in the patents referenced in Table 2 and listed above), as well as variants thereof, such as a variant having at least 98%, at least 95%, at least 90%, at least 80%, at least 70%, or at least 60% sequence identity with the native targeting moiety protein (e.g., at least about this amount of sequence identity to the GenBank Accession Nos. listed in Table 2 and listed above). In some examples, variant sequences retain substantially the same amount (or even more) of the native biological function of the targeting moiety protein, such as the ability to activate an intracellular signal cascade. However, variant targeting moiety molecules may in some examples retain little or no native biological activity, but retain the ability to bind the appropriate target (e.g., bind to the appropriate cell surface receptor or protein) with high specificity.

C. Linkers

Linking of a cargo moiety to a targeting moiety may be direct meaning that one portion of the cargo moiety is directly attached to a portion of the targeting moiety. For example, one end of the amino acid sequence of a cargo protein can be directly attached to an end of the amino acid sequence of the targeting moiety. For example, the C-terminus of the cargo protein can be linked to the N-terminus of the targeting moiety, or the C-terminus of the targeting moiety can be linked to the N-terminus of the cargo protein. Methods of generating such fusion proteins are routine in the art, for example using recombinant molecular biology methods.

In another example, the cargo moiety is linked to the targeting moiety indirectly through a linker. The linker can serve, for example, simply as a convenient way to link the two entities, as a means to spatially separate the two entities, to provide an additional functionality to the targeted cargo protein, or a combination thereof.

In general, the linker joining the targeting moiety and the cargo moiety can be designed to (1) allow the two molecules to fold and act independently of each other, (2) not have a propensity for developing an ordered secondary structure which could interfere with the functional domains of the two moieties, (3) have minimal hydrophobic or charged characteristic which could interact with the functional protein domains and/or (4) provide steric separation of the two regions. For example in some instances it may be desirable to spatially separate the targeting moiety and the cargo moiety to prevent the targeting moiety from interfering with the inhibitory activity of the targeted cargo moiety and/or the cargo moiety interfering with the targeting activity of the targeting moiety. The linker can also be used to provide, for example, lability to the connection between the targeting moiety and the cargo moiety, an enzyme cleavage site (for example a cleavage site for a protease), a stability sequence, a molecular tag, a detectable label, or various combinations thereof.

The linker can be bifunctional or polyfunctional, e.g. contains at least about a first reactive functionality at, or proximal to, a first end of the linker that is capable of bonding to, or being modified to bond to, the targeting moiety and a second reactive functionality at, or proximal to, the opposite end of the linker that is capable of bonding to, or being modified to bond to, the cargo moiety being modified. The two or more reactive functionalities can be the same (i.e. the linker is homobifunctional) or they can be different (i.e. the linker is heterobifunctional). A variety of bifunctional or polyfunctional cross-linking agents are known in the art that are suitable for use as linkers (for example, those commercially available from Pierce Chemical Co., Rockford, Ill.), such as avidin and biotin. Alternatively, these reagents can be used to add the linker to the targeting moiety and/or cargo moiety.

The length and composition of the linker can be varied considerably provided that it can fulfill its purpose as a molecular bridge. The length and composition of the linker are generally selected taking into consideration the intended function of the linker, and optionally other factors such as ease of synthesis, stability, resistance to certain chemical and/or temperature parameters, and biocompatibility. For example, the linker should not significantly interfere with the ability of the targeting moiety to target the targeted cargo protein to a cancer stem cell, or with the activity of the targeted cargo protein relating to activation, pore-forming ability, or toxin activity.

Linkers suitable for use may be branched, unbranched, saturated, or unsaturated hydrocarbon chains, as well as peptides as noted above. Furthermore, if the linker is a peptide, the linker can be attached to the targeting moiety and/or the cargo moiety using recombinant DNA technology. Such methods are well-known in the art and details of this technology can be found, for example, in Sambrook et al., supra.

In one example, the linker is a branched or unbranched, saturated or unsaturated, hydrocarbon chain having from 1 to 100 carbon atoms, wherein one or more of the carbon atoms is optionally replaced by —O— or —NR— (wherein R is H, or C1 to C6 alkyl), and wherein the chain is optionally substituted on carbon with one or more substituents selected from the group of (C1-C6) alkoxy, (C3-C6) cycloalkyl, (C1-C6) alkanoyl, (C1-C6) alkanoyloxy, (C1-C6) alkoxycarbonyl, (C1-C6) alkylthio, amide, azido, cyano, nitro, halo, hydroxy, oxo (═O), carboxy, aryl, aryloxy, heteroaryl, and heteroaryloxy.

Examples of suitable linkers include, but are not limited to, peptides having a chain length of 1 to 500 amino acid residues (such as 1 to 100, 1 to 50, 6 to 30, such as less than 30 amino acids). Typically surface amino acids in flexible protein regions include Gly, Asn and Ser. Other neutral amino acids, such as Thr and Ala, can also be used in the linker sequence. Additional amino acids can be included in the linker to provide unique restriction sites in the linker sequence to facilitate construction of the fusions. Other exemplary linkers include those derived from groups such as ethanolamine, ethylene glycol, polyethylene with a chain length of 6 to 100 carbon atoms, polyethylene glycol with 3 to 30 repeating units, phenoxyethanol, propanolamide, butylene glycol, butyleneglycolamide, propyl phenyl, and ethyl, propyl, hexyl, steryl, cetyl, and palmitoyl alkyl chains.

In one example, the linker is a branched or unbranched, saturated or unsaturated, hydrocarbon chain, having from 1 to 50 carbon atoms, wherein one or more of the carbon atoms is optionally replaced by —O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (═O), carboxy, aryl and aryloxy.

In a specific example, the linker is a peptide having a chain length of 1 to 50 amino acid residues, such as 1 to 40, 1 to 20, or 5 to 10 amino acid residues.

Peptide linkers that are susceptible to cleavage by enzymes of the complement system, urokinase, tissue plasminogen activator, trypsin, plasmin, or another enzyme having proteolytic activity may be used in one example. According to another example, the targeted cargo protein includes a targeting moiety attached via a linker susceptible to cleavage by enzymes having a proteolytic activity such as a urokinase, a tissue plasminogen activator, plasmin, thrombin or trypsin. In addition, targeting moieties may be attached to the cargo moiety via disulfide bonds (for example, the disulfide bonds on a cysteine molecule). Since many tumors naturally release high levels of glutathione (a reducing agent) this can reduce the disulfide bonds with subsequent release of the cargo moiety at the site of delivery.

In one example, the targeted cargo protein includes a targeting moiety linked by a cleavable linker region. In another example, the cleavable linker region is a protease-cleavable linker, although other linkers, cleavable for example by small molecules, may be used. Examples of protease cleavage sites are those cleaved by factor Xa, thrombin and collagenase. In one example, the protease cleavage site is one that is cleaved by a protease that is associated with a disease. In another example, the protease cleavage site is one that is cleaved by a protease that is up-regulated or associated with cancers in general. Examples of such proteases are uPA, the matrix metalloproteinase (MMP) family, the caspases, elastase, prostate specific antigen (PSA, a serine protease), and the plasminogen activator family, as well as fibroblast activation protein. In still another example, the cleavage site is cleaved by a protease secreted by cancer-associated cells. Examples of these proteases include matrixmetalloproteases, elastase, plasmin, thrombin, and uPA. In another example, the protease cleavage site is one that is up-regulated or associated with a specific cancer. The precise sequences are available in the art and the skilled person will have no difficulty in selecting a suitable cleavage site. By way of example, the protease cleavage region targeted by Factor Xa is I E G R. The protease cleavage region targeted by enterokinase is D D D D K. The protease cleavage region targeted by thrombin is L V P R G. In one example, the cleavable linker region is one which is targeted by endocellular proteases.

As known in the art, the attachment of a linker to cargo moiety (or of a linker element to a cleavable element, or a cleavable element to another cargo moiety) need not be a particular mode of attachment or reaction.

D. Exemplary Cargo Moiety/Targeting Moiety Combinations

Any combination of cargo moiety and targeting moiety can be used. In this section exemplary combinations of targeting moieties and cargo moieties are provided. In all examples that targeting moiety can be an antibody that specifically binds to a target, such as a fully humanized antibody.

GMCSF can be used as a targeting moiety and linked to pro-apoptotic BCL-2 proteins, such as BAX, BAD, BAT, BAK, BIK, BOK, BID, BIM, BMF and BOK, as well as toxins such as aerolysin, proaerolysin or Pseudomonas exotoxin. For example, GMCSF or fragments of GMCSF that bind to the GMCSF receptor can be used. Additionally, multiple cargo moieties can be linked to GMCSF or multiple GMCSF proteins can be linked to cargo moieties.

IL-4 (including IL-4 circularly permuted ligands and other IL-4 receptor binding proteins such as IL-13) is another targeting moiety that can be linked to BCL-2 family proteins, such as BAX, BAD, BAT, BAK, BIK, BOK, BID BIM, BMF and BOK, or a toxin such as aerolysin, proaerolysin, Pseudomonas exotoxin, or combinations thereof. Any form or derivative of IL-4 can be used as the targeting moiety. For example, IL-4 or fragments of IL-4 that bind to the IL-4 receptor can be used. Additionally, multiple cargo moieties can be linked to IL-4 or multiple IL-4 proteins can be linked to cargo moieties.

IL-2 is another targeting moiety that can be linked to BCL-2 family proteins, such as BAX, BAD, BAT, BAK, BIK, BOK, BID BIM, BMF and BOK, or a toxin such as aerolysin, proaerolysin, Pseudomonas exotoxin or combinations thereof. Any form or derivative of IL-2 can be used as the targeting moiety. For example, IL-2 or fragments of IL-2 that bind to the IL-2 receptor can be used. Additionally, multiple cargo moieties can be linked to IL-2 or multiple IL-2 proteins can be linked to cargo moieties.

An antibody that binds to tenascin is another targeting moiety that can be linked to BCL-2 family proteins, such as BAX, BAD, BAT, BAK, BIK, BOK, BID BIM, BMF and BOK, or a toxin such as bouganin, aerolysin, proaerolysin, Pseudomonas exotoxin or combinations thereof. Any fragment, form or derivative of the anti-tenascin antibody can be used as the targeting moiety. Additionally, multiple cargo moieties can be linked to the anti-tenascin antibody.

An antibody that binds to EpCAM is another targeting moiety that can be linked to BCL-2 family proteins, such as BAX, BAD, BAT, BAK, BIK, BOK, BID BIM, BMF and BOK, or a toxin such as bouganin, aerolysin, proaerolysin, Pseudomonas exotoxin or combinations thereof. Any fragment, form or derivative of the anti-EpCAM antibody can be used as the targeting moiety. Additionally, multiple cargo moieties can be linked to the anti-EpCAM antibody.

An antibody that binds to CD22 is another targeting moiety that can be linked to BCL-2 family proteins, such as BAX, BAD, BAT, BAK, BIK, BOK, BID BIM, BMF and BOK, or a toxin such as bouganin, aerolysin, proaerolysin, Pseudomonas exotoxin, or RNAse A or combinations thereof. Any fragment, form or derivative of the anti-CD22 antibody can be used as the targeting moiety. Additionally, multiple cargo moieties can be linked to the anti-CD22 antibody.

An antibody that binds to mesothelin is another targeting moiety that can be linked to BCL-2 family proteins, such as BAX, BAD, BAT, BAK, BIK, BOK, BID BIM, BMF and BOK, or a toxin such as bouganin, aerolysin, proaerolysin, Pseudomonas exotoxin, or combinations thereof. Any fragment, form or derivative of the anti-mesothelin antibody can be used as the targeting moiety. Additionally, multiple cargo moieties can be linked to the anti-mesothelin antibody.

An antibody that binds to PSMA is another targeting moiety that can be linked to BCL-2 family proteins, such as BAX, BAD, BAT, BAK, BIK, BOK, BID BIM, BMF and BOK, a toxin such as bouganin, aerolysin, proaerolysin, Pseudomonas exotoxin, thapsigargin, a chemotherapeutic agent, or combinations thereof. Any fragment, form or derivative of the anti-PSMA antibody can be used as the targeting moiety. Additionally, multiple cargo moieties can be linked to the anti-PSMA antibody.

EGF is another targeting moiety that can be linked to BCL-2 family moieties, such as BAX, BAD, BAT, BAK, BIK, BOK, BID BIM, BMF and BOK, or a toxin such as aerolysin, proaerolysin, Pseudomonas exotoxin or combinations thereof. Any form or derivative of EGF can be used as the targeting moiety. For example, EGF or fragments of EGF that bind to the EGF receptor can be used. Additionally, multiple cargo moieties can be linked to EGF or multiple EGF proteins can be linked to cargo moieties.

A circularly permuted ligand, for example a circularly permuted ligand derived from IL-4, IL-2, IL-3, IL-5, IL-10, IL-13, EGF, granulocyte colony stimulating factor (G-CSF) or granulocyte/macrophage colony stimulating factor (GMCSF) can be linked to a BCL-2 family protein, such as BAX, BAD, BAT, BAK, BIK, BOK, BID BIM, BMF and BOK, bouganin, aerolysin, proaerolysin, Pseudomonas exotoxin or combinations thereof. Any form or derivative of circularly permuted ligand can be used as the targeting moiety. Additionally, multiple cargo moieties can be linked to a circularly permuted ligand or multiple circularly permuted ligand proteins can be linked to cargo moieties.

Table 3 lists additional exemplary combinations of targeting moieties and cargo moieties. Each “X” indicates an exemplary targeted cargo protein.

TABLE 3
Exemplary targeted cargo proteins

| | Targeting Moiety | | | | | | | | |
|---|---|---|---|---|---|---|---|---|---|
| Cargo Moiety | Mesothelin | PSMA | CD22 | EpCAM | IL-2 | IL-4 | EGF | GMCSF | Tenascin |
| Aerolysin | | | | | X | | | | |
| Proaerolysin | | | | | X | | X | | |
| Pseudomonas | X | | X | X | | X | X | | X |
| exotoxin | | | | | | | | | |
| BAD | | | | | | X | | X | |
| Bouganin | | | | X | | | | | X |
| RNAseA | | | X | | | | | | |
| Thapsigarin | | X | | | | | | | |

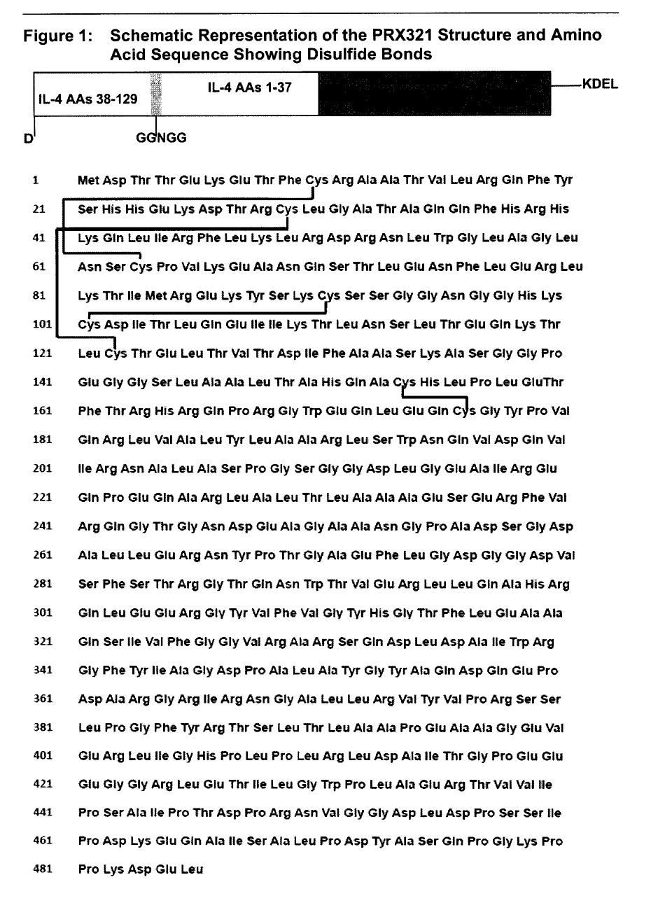

In some instances specific targeted cargo proteins are desired. In Table 3 an “X” indicates that the specific targeting moiety identified linked to the specific cargo moiety is desirable. Exemplary targeted cargo proteins include circularly permuted IL-4-Pseudomonas exotoxin (see U.S. Pat. No. 6,011,002 and the amino acid sequence of one embodiment shown in FIG. 1 and SEQ ID NO: 1), IL-2-aerolysin (see WO 2007/140618), IL-2-proaerolysin (see WO 2007/140618), EGF-proaerolysin, IL-4-BAD, anti-EpCAM-PE, anti-EpCAM-bouganin, GMCSF-BAD, anti-mesothelin antibody-PE, anti-CD22-PE, anti-CD22-RNase A, and anti-PSMA-thapsigargin.

### IV. Making Targeted Cargo Proteins

Targeted cargo proteins can be prepared by many routine methods as known in the art. Targeted cargo proteins, as well as modifications thereto, can be made, for example, by engineering the nucleic acid encoding the targeted cargo protein using recombinant DNA technology or by peptide synthesis. Modifications to the targeted cargo protein may be made, for example, by modifying the targeted cargo protein polypeptide itself, using chemical modifications and/or limited proteolysis. Combinations of these methods may also be used to prepare the targeted cargo proteins.

Methods of cloning and expressing proteins are well-known in the art, detailed descriptions of techniques and systems for the expression of recombinant proteins can be found, for example, in Current Protocols in Protein Science (Coligan, J. E., et al., Wiley & Sons, New York). Those skilled in the art will understand that a wide variety of expression systems can be used to provide the recombinant protein. Accordingly, the targeted cargo proteins can be produced in a prokaryotic host (e.g., E. coli, A. salmonicida or B. subtilis) or in a eukaryotic host (e.g., Saccharomyces or Pichia; mammalian cells, e.g., COS, NIH 3T3, CHO, BHK, 293, or HeLa cells; or insect cells). The targeted cargo proteins can be purified from the host cells by standard techniques known in the art.

Sequences for various exemplary cargo moieties and targeting moieties are provided in the Tables 1 and 2. Variants and homologs of these sequences can be cloned, if an alternative sequence is desired, using standard techniques [see, for example, Ausubel et al., Current Protocols in Molecular Biology, Wiley & Sons, NY (1997 and updates); Sambrook et al., supra]. For example, the nucleic acid sequence can be obtained directly from a suitable organism, such as Aeromonas hydrophila, by extracting mRNA and then synthesizing cDNA from the mRNA template (for example by RT-PCR) or by PCR-amplifying the gene from genomic DNA. Alternatively, the nucleic acid sequence encoding either the targeting moiety or the cargo moiety can be obtained from an appropriate cDNA library by standard procedures. The isolated cDNA is then inserted into a suitable vector, such as a cloning vector or an expression vector.

Mutations (if desired) can be introduced at specific, pre-selected locations by in vitro site-directed mutagenesis techniques well-known in the art. Mutations can be introduced by deletion, insertion, substitution, inversion, or a combination thereof, of one or more of the appropriate nucleotides making up the coding sequence.

The expression vector can further include regulatory elements, such as transcriptional elements, required for efficient transcription of the targeted cargo protein-encoding sequences. Examples of regulatory elements that can be incorporated into the vector include, but are not limited to, promoters, enhancers, terminators, and polyadenylation signals. Vectors that include a regulatory element operatively linked to a nucleic acid sequence encoding a genetically engineered targeted cargo protein can be used to produce the targeted cargo protein.

The expression vector may additionally contain heterologous nucleic acid sequences that facilitate the purification of the expressed targeted cargo protein, such as affinity tags such (e.g., metal-affinity tags, histidine tags, avidin/streptavidin encoding sequences, glutathione-S-transferase (GST) encoding sequences, and biotin encoding sequences). In one example, such tags are attached to the N- or C-terminus of a targeted cargo protein, or can be located within the targeted cargo protein. The tags can be removed from the expressed targeted cargo protein prior to use according to methods known in the art. Alternatively, the tags can be retained on the targeted cargo protein, providing that they do not interfere with the ability of the targeted cargo protein to target and kill (or decrease growth of) cancer stem cells.

As an alternative to a directed approach to introducing mutations into naturally occurring pore-forming proteins, a cloned gene expressing a pore-forming protein can be subjected to random mutagenesis by techniques known in the art. Subsequent expression and screening of the mutant forms of the protein thus generated would allow the identification and isolation of targeted cargo moieties.

The targeted cargo proteins can also be prepared as fragments or fusion proteins. A fusion protein is one which includes a targeted cargo protein linked to other amino acid sequences that do not inhibit the ability of the targeted cargo protein to selectively target and inhibit cancer stem cell growth or kill cancer stem cells. In an alternative example, the other amino acid sequences are short sequences of, for example, up to about 5, about 6, about 7, about 8, about 9, about 10, about 20, about 30, about 50 or about 100 amino acid residues in length. These short sequences can be linker sequences as described above.

Methods for making fusion proteins are well known to those skilled in the art. For example U.S. Pat. No. 6,057,133 discloses methods for making fusion molecules composed of human interleukin-3 (hIL-3) variant or mutant proteins functionally joined to a second colony stimulating factor, cytokine, lymphokine, interleukin, hematopoietic growth factor or IL-3 variant. U.S. Pat. No. 6,072,041 to Davis et al. discloses the generation of fusion proteins comprising a single chain Fv molecule directed against a transcytotic receptor covalently linked to a therapeutic protein.

The targeted cargo protein can include one or more linkers, as well as other moieties, as desired. These can include a binding region, such as avidin or an epitope, or a tag such as a polyhistidine tag, which can be useful for purification and processing of the fusion protein. In addition, detectable markers can be attached to the fusion protein, so that the traffic of the fusion protein through a body or cell can be monitored conveniently. Such markers include radionuclides, enzymes, fluorophores, chromophores, and the like.

One of ordinary skill in the art will appreciate that the DNA can be altered in numerous ways without affecting the biological activity of the encoded protein. For example, PCR can be used to produce variations in the DNA sequence which encodes a targeted cargo protein. Such variations in the DNA sequence encoding a targeted cargo protein can be used to optimize for codon preference in a host cell used to express the protein, or may contain other sequence changes that facilitate expression.

A covalent linkage of a targeting moiety directly to a cargo moiety or via a linker may take various forms as is known in the art. For example, the covalent linkage may be in the form of a disulfide bond. The DNA encoding one of the components can be engineered to contain a unique cysteine codon. The second component can be derivatized with a sulfhydryl group reactive with the cysteine of the first component. Alternatively, a sulfhydryl group, either by itself or as part of a cysteine residue, can be introduced using solid phase polypeptide techniques. For example, the introduction of sulfhydryl groups into peptides is described by Hiskey (Peptides 3:137, 1981).

Proteins also can be chemically modified by standard techniques to add a sulfhydryl group. For example, Traut's reagent (2-iminothiolane-HCl) (Pierce Chemicals, Rockford, Ill.) can be used to introduce a sulfhydryl group on primary amines, such as lysine residues or N-terminal amines. A protein or peptide modified with Traut's reagent can then react with a protein or peptide which has been modified with reagents such as N-succinimidyl 3-(2-pyridyldithio)propionate (SPDP) or succinimidyl 4-(N-maleimidomethyl)cyclohexane-1-carboxylate (SMCC) (Pierce Chemicals, Rockford, Ill.).

The components can also be joined using the polymer, monomethoxy-polyethylene glycol (mPEG), as described in Maiti et al., Int. J. Cancer Suppl., 3:17-22, 1988.

The targeting moiety and the cargo moiety can also be conjugated through the use of standard conjugation chemistries as is known in the art, such as carbodiimide-mediated coupling (for example, DCC, EDC or activated EDC), and the use of 2-iminothiolane to convert epsilon amino groups to thiols for crosslinking and m-maleimidobenzoyl-n-hydroxysuccinimidyl ester (MBS) as a crosslinking agent.

### V. Testing Targeted Cargo Proteins

Targeted cargo proteins can be tested using standard techniques known in the art. Exemplary methods of testing candidate targeted cargo proteins are provided below and in the examples included herein. One of ordinary skill in the art will understand that other methods of testing the targeted cargo proteins are known in the art and are also suitable for testing candidate targeted cargo proteins. For example, methods known in the art for testing for anti-tumor activity can be used. The targeted cargo proteins can initially be screened against a panel of cancer stem cell lines. A cell proliferation assay, such as the WST-1 kit sold by Roche, can be used. Potency can be evaluated using different drug concentrations in the presence or absence of agents that inhibit cancer cells or sensitize cancer stem cells. Selected drug candidates from the initial cancer stem cell screen can be further characterized through additional in vitro assays and in relevant xenograft models to examine anti-tumor activity.

A. In Vitro

Targeted cargo proteins can be tested for their ability to kill cancer stem cells or significantly reduce or inhibit the growth of cancer stem cells using known methods. For example, the ability of the targeted cargo proteins to kill or inhibit growth of cells can be assayed in vitro using suitable cells, typically a cell line expressing the target or a stem cancer cell. In general, cells of the selected test cell line are grown to an appropriate density and the candidate targeted cargo protein is added. The targeted cargo protein can be added to the culture at around at least 1 ng/mL, at least 1 μg/mL, or at least 1 mg/mL, such as from about 0.01 μg/mL to about 1 mg/mL, from about 0.10 μg/mL to about 0.5 mg/mL, from about 1 μg/mL to about 0.4 mg/mL. In some examples, serial dilutions are tested. After an appropriate incubation time (for example, about 48 to 72 hours), cell survival or growth is assessed. Methods of determining cell survival are well known in the art and include, but are not limited to, the resazurin reduction test (see Fields & Lancaster Am. Biotechnol. Lab., 11:48-50, 1993; O'Brien et al., Eur. J. Biochem., 267:5421-5426, 2000 and U.S. Pat. No. 5,501,959), the sulforhodamine assay (Rubinstein et al., J. Natl. Cancer Inst., 82:113-118, 1999) or the neutral red dye test (Kitano et al., Euro. J. Clin. Investg., 21:53-58, 1991; West et al., J. Investigative Derm., 99:95-100, 1992) or trypan blue assay. Numerous commercially available kits may also be used, for example the CellTiter 96®AQueous One Solution Cell Proliferation Assay (Promega). Cytotoxicity is determined by comparison of cell survival in the treated culture with cell survival in one or more control cultures, for example, untreated cultures and/or cultures pre-treated with a control compound (typically a known therapeutic), or other appropriate control. Targeted cargo proteins considered to be effective in killing or reducing the growth of cancer stem cells are capable of decreasing cell survival or growth, for example, by at least about 10%, at least about 20%, at least about 30%, at least about 40%, or at least about 50%.

In some examples the targeted cargo protein can be not significantly toxic to non-cancer stem cells. For example, the targeted cargo protein when incubated at around at least 1 ng/mL, at least 1 μg/mL, or at least 1 mg/mL, such as from about 0.01 μg/mL to about 1 mg/mL, from about 0.10 μg/mL to about 0.5 mg/mL, from about 1 μg/mL to about 0.4 mg/mL in cell culture with cells not displaying the target (e.g., does not express IL-2R) will kill less than about 50%, less than about 40%, less than about 30%, less than about 20%, or less than about 10% of the non-cancer stem cells. In some examples, the targeted cargo protein when incubated at around at least 1 ng/mL, at least 1 μg/mL, or at least 1 mg/mL, such as from about 0.01 μg/mL to about 1 mg/mL, from about 0.10 μg/mL to about 0.5 mg/mL, from about 1 μg/mL to about 0.4 mg/mL in cell culture with cells not displaying the target (e.g., does not express IL-2R) will have at least a 10-fold greater LD₅₀ toward the non-cancer stem cells, such as an at least 20-fold greater, at least 50-fold greater, or at least 100-fold greater LD₅₀ toward the non-cancer stem cells.

In some examples targeted cargo proteins include a toxin that contains one or more modifications to an activation sequence. These activatable targeted cargo proteins can be tested for their ability to be cleaved by the appropriate activating agent according to methods known in the art. For example, if the one or more modifications result in the addition of one or more protease cleavage sites, the targeted cargo protein can be incubated with varying concentrations of the appropriate protease(s). The incubation products can be electrophoresed on SDS-PAGE gels and cleavage of the targeted cargo protein can be assessed by examining the size of the polypeptide on the gel.

In order to determine if the activatable targeted cargo proteins that have been incubated with protease retain pore-forming activity, and thus the ability to kill cells, after incubation with the protease, the reaction products can be tested in a hemolysis assay as is known in the art. An example of a suitable assay is described in Howard and Buckley, J. Bacteriol., 163:336-40, 1985, which is herein incorporated by reference.

Targeted cargo proteins that confer selectivity for a specific type of cancer may be tested for their ability to target that specific cancer cell type. For example, a targeted cargo protein comprising an IL-4 that targets cancer stem cells displaying IL-4R can be assessed for its ability to selectively target cancer stem cells by comparing the ability of the targeted cargo protein to kill cancer stem cells to its ability to kill a normal cell, or a different type of cancer cell (e.g., one that does not express IL-4R). Alternatively, flow cytometric methods, as are known in the art, may be used to determine if a targeted cargo protein comprising an IL-4 targeting moiety is able to selectively target a specific type of cancer stem cell. Binding of a labeled antibody to the bound targeted cargo protein will indicate binding of the targeted cargo protein to the target.

A variety of cancer cell-lines suitable for testing the candidate targeted cargo proteins are known in the art and many are commercially available (for example, from the American Type Culture Collection, Manassas, Va.). In one example, in vitro testing of the candidate compounds is conducted in a human cancer cell-line. In another example, cancer stem cells are isolated and cultured as described in US Patent Application No. 2007/0292389 to Stassi et al. The cultured stem cells are used to test the activity of the targeted cargo protein. Initial testing of the targeting moiety can be performed by linking the targeting moiety to a detectable label such as a fluorescent label and contacting a sample known to contain the appropriate cancer stem cells with the targeting moiety and observing the associated fluorescent label bound to the cancer stem cell.

Additional in vitro testing of targeted cargo proteins can be accomplished using cell lines that have been engineered to express the desired target. An antibody specific for the target can be used to ensure that the target is being expressed. Upon binding to the cell expressing the target, the targeted cargo protein may cause cell lysis which can be detected using methods known in the art.

B. In Vivo

The ability of the targeted cargo proteins to kill tumor cells in vivo can be determined in an appropriate animal model using standard techniques known in the art (see, for example, Enna, et al., Current Protocols in Pharmacology, J. Wiley & Sons, Inc., New York, N.Y.).

Current animal models for screening anti-tumor compounds include xenograft models, in which a human tumor has been implanted into an animal. Using these techniques cancer stem cells can be transplanted and the presence, size and morphology of the resulting tumor can be assessed. Examples of xenograft models of human cancer include, but are not limited to, human solid tumor xenografts, implanted by sub-cutaneous injection or implantation and used in tumor growth assays; human solid tumor isografts, implanted by fat pad injection and used in tumor growth assays; human solid tumor orthotopic xenografts, implanted directly into the relevant tissue and used in tumor growth assays; experimental models of lymphoma and leukemia in mice, used in survival assays, and experimental models of lung metastasis in mice. In addition to the implanted human tumor cells, the xenograft models can further comprise transplanted human peripheral blood leukocytes, which allow for evaluation of the anti-cancer immune response.

Alternatively, murine cancer models can be used for screening anti-tumor compounds. Examples of appropriate murine cancer models are known in the art and include, but are not limited to, implantation models in which murine cancer cells are implanted by intravenous, subcutaneous, fat pad or orthotopic injection; murine metastasis models; transgenic mouse models; and knockout mouse models.

For example, the targeted cargo proteins can be tested in vivo on solid tumors using mice that are subcutaneously grafted bilaterally with 30 to 60 mg of a tumor fragment, or implanted with an appropriate number of cancer stem cells (e.g., at least 10³, at least 10⁴, or at least at least 10⁶ cancer stem cells, such as from about 10 to about 10⁵, from about 50 to about 10⁴, or from about 75 to about 10³), on day 0. The animals bearing tumors are randomized before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor, and the animals are observed every day. The targeted cargo proteins can be administered to the animals, for example, by i.p. injection, intravenous injection, direct injection into the tumor (or into the organ having the tumor), or bolus infusion. The amount of targeted cargo protein that is injected can be determined using the in vitro testing results described above. For example, at least about 1 ng/kg body weight, at least 1 μg/kg body weight, or at least 1 mg/kg body weight, such as from about 0.01 μg/kg body weight to about 1 mg/kg body weight, from about 0.10 μg/kg body weight to about 1.0 g/kg body weight, from about 1 mg/kg body weight to about 4 mg/kg body weight. The different animal groups are weighed about 3 or 4 times a week until the maximum weight loss is attained, after which the groups are weighed at least about once a week until the end of the trial.

The tumors are measured after a pre-determined time period, or they can be monitored continuously by measuring about 2 or 3 times a week until the tumor reaches a pre-determined size and/or weight, or until the animal dies if this occurs before the tumor reaches the pre-determined size/weight. The animals are then sacrificed and the tissue histology, size and/or proliferation of the tumor assessed.

Orthotopic xenograft models are an alternative to subcutaneous models and may more accurately reflect the cancer development process. In this model, tumor cells are implanted at the site of the organ of origin and develop internally. Daily evaluation of the size of the tumors is thus more difficult than in a subcutaneous model. A recently developed technique using green fluorescent protein (GFP) expressing tumors in non-invasive whole-body imaging can help to address this issue (Yang et al., Proc. Nat. Aca. Sci., 1206-1211, 2000). This technique utilizes human or murine tumors that stably express very high levels of green fluorescent protein (GFP). The GFP expressing tumors can be visualized by means of externally placed video detectors, allowing for monitoring of details of tumor growth, angiogenesis and metastatic spread. Angiogenesis can be measured over time by monitoring the blood vessel density within the tumor(s). The use of this model thus allows for simultaneous monitoring of several features associated with tumor progression and has high preclinical and clinical relevance.

For the study of the effect of the compositions on leukemias, the animals are grafted with a particular number of cells, and the anti-tumor activity is determined by the increase in the survival time of the treated mice relative to the controls.

To study the effect of a particular targeted cargo protein on tumor metastasis, tumor cells are typically treated with the composition ex vivo and then injected into a suitable test animal. The spread of the tumor cells from the site of injection is then monitored over a suitable period of time.

Targeted cargo proteins that are sufficiently effective at inhibiting cancer stem cell growth (as evidenced by in vitro cell survival assays, metastasis inhibition assays, and/or xenograph model systems) can be chosen for use in humans. Targeted cargo proteins can also be chosen for trial and eventual therapeutic use in humans based upon their relative toxicity at the potential therapeutic dosage range indicated by the assays. Therapeutic dosages and toxicity are further described below.

### VI. Therapeutic Uses

The targeted cargo proteins described herein can be used for a variety of therapeutic purposes. Prior to administration for therapeutic purposes the targeted cargo protein may need to be modified or adapted for the particular purpose, for example the concentration of targeted cargo protein needed for whole body administration may differ from that used for local administration. Similarly, the toxicity of the therapeutic may change depending upon the mode of administration and overall composition being used (e.g., buffer, diluent, additional chemotherapeutic, etc.).

A. Toxicity

Therapeutic proteins may elicit some level of antibody response when administered to a subject, which in some cases may lead to undesirable side effects. Therefore, if necessary, the antigenicity of the targeted cargo proteins can be assessed as known in the art and described below. In addition, methods to reduce potential antigenicity are described.

In vivo toxic effects of the targeted cargo proteins can be evaluated by measuring their effect on animal body weight during treatment and by performing hematological profiles and liver enzyme analysis after the animal has been sacrificed. The general toxicity of the targeted cargo proteins can be tested according to methods known in the art. For example, the overall systemic toxicity of the targeted cargo proteins can be tested by determining the dose that kills 100% of mice (i.e. LD100) following a single intravenous injection. Doses that were at least about 2, 5, or 10-fold less than the LD100 or LD50 can be selected for administration into other mammals, such as a human.

The kinetics and magnitude of the antibody response to the targeted cargo proteins described herein can be determined, for example, in immunocompetent mice and can be used to facilitate the development of a dosing regimen that can be used in an immunocompetent human. Immunocompetent mice such as the strain C57-BL6 are administered intravenous doses of targeted cargo protein. The mice are sacrificed at varying intervals (e.g. following single dose, following multiple doses) and serum obtained. An ELISA-based assay can be used to detect the presence of anti-targeted cargo protein antibodies.

To decrease antigenicity of targeted cargo proteins the native binding domain of the toxin used as the cargo moiety can be functionally deleted and replaced, for example with a targeting moiety to make the targeted cargo protein. The antigenicity of such targeted cargo proteins can be determined following exposure to varying schedules of the targeted cargo protein which lack portions of the native binding domain using the methods described above. Targeted cargo proteins that utilize fully humanized antibodies can also be used to minimize antigenicity.

Another method that can be used to allow continued treatment with targeted cargo proteins is to use sequentially administered alternative targeted cargo proteins derived from other cargo proteins with non-overlapping antigenicity. For example, a targeted cargo protein derived from proaerolysin can be used alternately with a targeted cargo protein derived from Clostridium septicum alpha toxin or Bacillus thuringiensis delta-toxin. All of these targeted cargo proteins would target cancer stem cells, but would not be recognized or neutralized by the same antibodies.

Serum samples from these mice can be assessed for the presence of anti-targeted cargo protein antibodies as known in the art. As another example, epitope mapping can also be used to determine antigenicity of proteins as described in Stickler, et al., J. Immunotherapy, 23:654-660, 2000. Briefly, immune cells known as dendritic cells and CD4+ T cells are isolated from the blood of community donors who have not been exposed to the protein of interest. Small synthetic peptides spanning the length of the protein are then added to the cells in culture. Proliferation in response to the presence of a particular peptide suggests that a T cell epitope is encompassed in the sequence. This peptide sequence can subsequently be deleted or modified in the targeted cargo protein thereby reducing its antigenicity.

B. Pharmaceutical Compositions

Pharmaceutical compositions can include one or more targeted cargo proteins and one or more non-toxic pharmaceutically acceptable carriers, diluents, excipients and/or adjuvants. If desired, other active ingredients may be included in the compositions. As indicated above, such compositions are suitable for use in the treatment of cancer. The term “pharmaceutically acceptable carrier” refers to a carrier medium which does not interfere with the effectiveness of the biological activity of the active ingredients and which is not toxic to the host or patient. Representative examples are provided below.

The pharmaceutical compositions may comprise, for example, from about 1% to about 95% of a targeted cargo protein. Compositions formulated for administration in a single dose form may comprise, for example, about 20% to about 90% of the targeted cargo proteins, whereas compositions that are not in a single dose form may comprise, for example, from about 5% to about 20% of the targeted cargo proteins. Concentration of the targeted cargo protein in the final formulation can be at least 1 ng/mL, such as at least 1 μg/mL or at least 1 mg/mL. For example, the concentration in the final formulation can be between about 0.01 μg/mL and about 1,000 μg/mL. In one example, the concentration in the final formulation is between about 0.01 mg/mL and about 100 mg/mL.

The composition can be a liquid solution, suspension, emulsion, sustained release formulation, or powder. The composition can be formulated as a suppository, with traditional binders and carriers such as triglycerides.

The targeted cargo proteins can be delivered along with a pharmaceutically acceptable vehicle. In one example, the vehicle may enhance the stability and/or delivery properties. Thus, the disclosure also provides for formulation of the targeted cargo protein with a suitable vehicle, such as an artificial membrane vesicle (including a liposome, noisome, nanosome and the like), microparticle or microcapsule, or as a colloidal formulation that comprises a pharmaceutically acceptable polymer. The use of such vehicles/polymers may be beneficial in achieving sustained release of the targeted cargo proteins. Alternatively, or in addition, the targeted cargo protein formulations can include additives to stabilize the protein in vivo, such as human serum albumin, or other stabilizers for protein therapeutics known in the art. Targeted cargo protein formulations can also include one or more viscosity enhancing agents which act to prevent backflow of the formulation when it is administered, for example by injection or via catheter. Such viscosity enhancing agents include, but are not limited to, biocompatible glycols and sucrose.

Pharmaceutical compositions formulated as aqueous suspensions contain the active compound(s) in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, or one or more coloring agents.

Pharmaceutical compositions can be formulated as oily suspensions by suspending the active compound(s) in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Compositions can be preserved by the addition of an anti-oxidant such as ascorbic acid.

The pharmaceutical compositions can be formulated as a dispersible powder or granules, which can subsequently be used to prepare an aqueous suspension by the addition of water. Such dispersible powders or granules provide the active ingredient in admixture with one or more dispersing or wetting agents, suspending agents and/or preservatives. Suitable dispersing or wetting agents and suspending agents are exemplified by those already mentioned above.

Pharmaceutical compositions can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate.

The pharmaceutical compositions containing one or more targeted cargo proteins can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

In one example, the targeted cargo protein is conjugated to a water-soluble polymer, e.g., to increase stability or circulating half life or reduce immunogenicity. Clinically acceptable, water-soluble polymers include, but are not limited to, polyethylene glycol (PEG), polyethylene glycol propionaldehyde, carboxymethylcellulose, dextran, polyvinyl alcohol (PVA), polyvinylpyrrolidone (PVP), polypropylene glycol homopolymers (PPG), polyoxyethylated polyols (POG) (e.g., glycerol) and other polyoxyethylated polyols, polyoxyethylated sorbitol, or polyoxyethylated glucose, and other carbohydrate polymers. Methods for conjugating polypeptides to water-soluble polymers such as PEG are described, e.g., in U.S. patent Pub. No. 20050106148 and references cited therein. In one example the polymer is a pH-sensitive polymers designed to enhance the release of drugs from the acidic endosomal compartment to the cytoplasm (see for example, Henry et al., Biomacromolecules 7(8):2407-14, 2006).

Targeted cargo proteins can also be administered in therapeutically effective amounts together with one or more anti-cancer therapeutics. The compound(s) can be administered before, during or after treatment with the anti-cancer therapeutic. An “anti-cancer therapeutic” is a compound, composition, or treatment (e.g., surgery) that prevents or delays the growth and/or metastasis of cancer cells. Such anti-cancer therapeutics include, but are not limited to, surgery (e.g., removal of all or part of a tumor), chemotherapeutic drug treatment, radiation, gene therapy, hormonal manipulation, immunotherapy (e.g., therapeutic antibodies and cancer vaccines) and antisense or RNAi oligonucleotide therapy. Examples of useful chemotherapeutic drugs include, but are not limited to, hydroxyurea, busulphan, cisplatin, carboplatin, chlorambucil, melphalan, cyclophosphamide, Ifosphamide, danorubicin, doxorubicin, epirubicin, mitoxantrone, vincristine, vinblastine, Navelbine® (vinorelbine), etoposide, teniposide, paclitaxel, docetaxel, gemcitabine, cytosine, arabinoside, bleomycin, neocarcinostatin, suramin, taxol, mitomycin C, Avastin, Herceptin®, fluorouracil, and temozolamide and the like. The compounds are also suitable for use with standard combination therapies employing two or more chemotherapeutic agents. It is to be understood that anti-cancer therapeutics includes novel compounds or treatments developed in the future.

The pharmaceutical compositions described above include one or more targeted cargo proteins in an amount effective to achieve the intended purpose. Thus the term “therapeutically effective dose” refers to the amount of the targeted cargo protein that ameliorates the symptoms of cancer. Determination of a therapeutically effective dose of a compound is well within the capability of those skilled in the art. For example, the therapeutically effective dose can be estimated initially either in cell culture assays, or in animal models, such as those described herein. Animal models can also be used to determine the appropriate concentration range and route of administration. Such information can then be used to determine useful doses and routes for administration in other animals, including humans, using standard methods known in those of ordinary skill in the art.

Therapeutic efficacy and toxicity can also be determined by standard pharmaceutical procedures such as, for example, by determination of the median effective dose, or ED₅₀ (i.e. the dose therapeutically effective in 50% of the population) and the median lethal dose, or LD₅₀ (i.e. the dose lethal to 50% of the population). The dose ratio between therapeutic and toxic effects is known as the “therapeutic index,” which can be expressed as the ratio, LD₅₀/ED₅₀. The data obtained from cell culture assays and animal studies can be used to formulate a range of dosage for human or animal use. The dosage contained in such compositions is usually within a range of concentrations that include the ED₅₀ and demonstrate little or no toxicity. The dosage varies within this range depending upon the dosage form employed, sensitivity of the subject, and the route of administration and the like. Exemplary dosage ranges that can be used include at least 1 ng/g tumor, at least 1 μg/g tumor, or at least 1 mg/g tumor, such as dosage ranges from about 0.01 μg/g tumor to about 50 μg/g tumor, from about 0.02 μg/g tumor to about 40 μg/g tumor, from about 0.02 μg/g tumor to about 35 μg/g tumor, 0.03 μg/g tumor to about 25 μg/g tumor, from about 0.04 μg/g tumor to about 20 μg/g tumor, from about 0.04 μg/g tumor to about 10 μg/g tumor, and from about 0.5 μg/g tumor to about 2 μg/g tumor.

One of ordinary skill in the art will appreciate that the dosage will depend, among other things, upon the type of targeted cargo protein being used and the type of cancer stem cell being treated.

C. Indications

The targeted cargo proteins can be used to treat, stabilize or prevent cancer. Targeted cargo proteins can also be used in the treatment of indolent cancers, recurrent cancers including locally recurrent, distantly recurrent and/or refractory cancers (i.e. cancers that have not responded to other anti-cancer treatments), metastatic cancers, locally advanced cancers and aggressive cancers. In these contexts, the targeted cargo proteins may exert either a cytotoxic or cytostatic effect resulting in, for example, a reduction in the number or growth of cancer stem cells, a reduction in the size of a tumor, the slowing or prevention of an increase in the size of a tumor, an increase in the disease-free survival time between the disappearance or removal of a tumor and its reappearance, prevention of an initial or subsequent occurrence of a tumor (e.g. metastasis), an increase in the time to progression, reduction of one or more adverse symptoms associated with a tumor, or an increase in the overall survival time of a subject having cancer.

Typically in the treatment of cancer, targeted cargo proteins are administered systemically to patients, for example, by bolus injection or continuous infusion into a patient's bloodstream. Alternatively, the targeted cargo proteins may be administered locally, at the site of a tumor (intratumorally). When a targeted cargo protein is administered intratumorally, the administration can be via any route, e.g., locally, regionally, focally, systemic, convection enhanced delivery or combinations thereof.

When used in conjunction with one or more known chemotherapeutic agents, the compounds can be administered prior to, or after, administration of the chemotherapeutic agents, or they can be administered concomitantly. The one or more chemotherapeutics may be administered systemically, for example, by bolus injection or continuous infusion, or they may be administered orally.

For administration to an animal, the pharmaceutical compositions can be formulated for administration by a variety of routes. For example, the compositions can be formulated for topical, rectal or parenteral administration or for administration by inhalation or spray. The term parenteral as used herein includes subcutaneous injections, intravenous, intramuscular, intrathecal, intrasternal injection or infusion techniques. Direct injection or infusion into a tumor is also contemplated. Convection enhanced delivery can also be used to administer the targeted cargo protein.

In one example, the targeted cargo protein can be injected into a subject having cancer, using an administration approach similar to the multiple injection approach of brachytherapy. For example, multiple aliquots of the purified targeted cargo protein in the form of a pharmaceutical composition or formulation and in the appropriate dosage units, may be injected using a needle. Alternative methods of administration of the targeted cargo proteins will be evident to one of ordinary skill in the art. Such methods include, for example, the use of catheters, or implantable pumps to provide continuous infusion of the targeted cargo protein to the subject in need of therapy.

As is known in the art, software planning programs can be used in combination with brachytherapy treatment and ultrasound, for example, for placement of catheters for infusing targeted cargo proteins to treat, for example, brain tumors or other localized tumors. For example, the positioning and placement of the needle can generally be achieved under ultrasound guidance. The total volume, and therefore the number of injections and deposits administered to a patient, can be adjusted, for example, according to the volume or area of the organ to be treated. An example of a suitable software planning program is the brachytherapy treatment planning program Variseed 7.1 (Varian Medical Systems, Palo Alto, Calif.). Such approaches have been successfully implemented in the treatment of prostate cancer among others.

If necessary to reduce a systemic immune response to the targeted cargo proteins, immunosuppressive therapies can be administered in combination with the targeted cargo proteins. Examples of immunosuppressive therapies include, but are not limited to, systemic or topical corticosteroids (Suga et al., Ann. Thorac. Surg., 73:1092-7, 2002), cyclosporin A (Fang et al., Hum. Gene Ther., 6:1039-44, 1995), cyclophosphamide (Smith et al., Gene Ther., 3:496-502, 1996), deoxyspergualin (Kaplan et al., Hum. Gene Ther., 8:1095-1104, 1997) and antibodies to T and/or B cells [e.g. anti-CD40 ligand, anti CD4 antibodies, anti-CD20 antibody (Rituximab)] (Manning et al., Hum. Gene Ther., 9:477-85, 1998). Such agents can be administered before, during, or subsequent to administration of the targeted cargo proteins. Such agents can be administered from about 10 mg/week to about 1000 mg/week, from about 40 mg/week to about 700 mg/week, or from about 200 mg/week to about 500 mg/week for 2, 3, 4, 5, 6, or 7 weeks. Courses of treatment can be repeated as necessary if the subject remains responsive (e.g., the symptoms of cancer are static or decreasing).

The targeted cargo protein can also be administered in combination with a sensitizing agent, such as a radio-sensitizers (see for example Diehn et al., J. Natl. Cancer Inst. 98:1755-7, 2006). Generally a sensitizing agent is any agent that increases the activity of a targeted cargo protein. For example, a sensitizing agent will increase the ability of a targeted cargo protein to inhibit cancer stem cell growth or kill cancer stem cells. Exemplary sensitizing agents include antibodies to IL-10, bone morphogenic proteins and HDAC inhibitors (see for example Sakariassen et al., Neoplasia 9(11):882-92, 2007). These sensitizing agents can be administered before or during treatment with the targeted cargo protein. Exemplary dosages of such sensitizing agents include at least 1 μg/mL, such as at least 10 μg/mL, at least 100 μg/mL, for example 5-100 μg/mL or 10-90 μg/mL. The sensitizing agents can be administered daily, three times a week, twice a week, once a week or once every two weeks. Sensitizing agent can also be administered after treatment with the targeted cargo protein is finished.

The targeted cargo proteins may be used as part of a neo-adjuvant therapy (to primary therapy), as part of an adjuvant therapy regimen, where the intention is to cure the cancer in a subject. The targeted cargo proteins can also be administered at various stages in tumor development and progression, including in the treatment of advanced and/or aggressive neoplasias (e.g., overt disease in a subject that is not amenable to cure by local modalities of treatment, such as surgery or radiotherapy), metastatic disease, locally advanced disease and/or refractory tumors (e.g., a cancer or tumor that has not responded to treatment).

“Primary therapy” refers to a first line of treatment upon the initial diagnosis of cancer in a subject. Exemplary primary therapies may involve surgery, a wide range of chemotherapies and radiotherapy. “Adjuvant therapy” refers to a therapy that follows a primary therapy and that is administered to subjects at risk of relapsing. Adjuvant systemic therapy is begun soon after primary therapy, for example 2, 3, 4, 5, or 6 weeks after the last primary therapy treatment to delay recurrence, prolong survival or cure a subject.

As noted above, it is contemplated that the targeted cargo proteins can be used alone or in combination with one or more other chemotherapeutic agents as part of an adjuvant therapy. Combinations of the targeted cargo proteins and standard chemotherapeutics may act to improve the efficacy of the chemotherapeutic and, therefore, can be used to improve standard cancer therapies. This application can be particularly important in the treatment of drug-resistant cancers which are not responsive to standard treatment.

The dosage to be administered is not subject to defined limits, but it will usually be an effective amount. The compositions may be formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. The unit dosage forms may be administered once or multiple unit dosages may be administered, for example, throughout an organ, or solid tumor. Examples of ranges for the targeted cargo protein(s) in each dosage unit are from about 0.0005 to about 100 mg, or more usually, from about 1.0 to about 1000 mg. Daily dosages of the targeted cargo proteins typically are at least 1 ng/kg of body weight, at least 1 μg/kg of body weight, at least 1 mg/kg of body weight, for example fall within the range of about 0.01 to about 100 mg/kg of body weight, in single or divided dose. However, it will be understood that the actual amount of the compound(s) to be administered will be determined by a physician, in the light of the relevant circumstances, including the condition to be treated, the chosen route of administration, the actual compound administered, the age, weight, and response of the individual patient, and the severity of the patient's symptoms. The above dosage range is given by way of example only and is not intended to limit the scope in any way. In some instances dosage levels below the lower limit of the aforesaid range may be more than adequate, while in other cases still larger doses may be employed without causing harmful side effects, for example, by first dividing the larger dose into several smaller doses for administration throughout the day.

The targeted cargo proteins can be used to treat and/or manage cancer, the methods include administering to a subject in need thereof a prophylactically or therapeutically effective regimen, the regimen comprising administering one or more therapies to the subject, wherein the regimen results in the stabilization or reduction in the cancer stem cell population and does not result in a reduction or only results in a small reduction of the circulating endothelial cell population and/or the circulating endothelial progenitor population. In one example, the regimen achieves a 5%-40%, a 10%-60%, or a 20 to 99% reduction in the cancer stem cell population and/or less than a 25%, less than a 15%, or less than a 10% reduction in the circulating endothelial cell population. In another example, the regimen achieves a 5%-40%, a 10%-60%, or a 20 to 99% reduction in the cancer stem cell population and/or less than a 25%, less than a 15%, or less than a 10% reduction in the circulating endothelial progenitor population. In another example, the regimen achieves a 5%-40%, a 10%-60%, or a 20 to 99% reduction in the cancer stem cell population and/or less than a 25%, less than a 15%, or less than a 10% reduction in the circulating endothelial cell population and the circulating endothelial progenitor population. In a specific example, the stabilization or reduction in the cancer stem cell population is achieved after two weeks, a month, two months, three months, four months, six month, nine months, 1 year, 2 years, 3 years, 4 years or more of administration of one or more of the therapies. In a particular example, the stabilization or reduction in the cancer stem cell population can be determined using any method known in the art. In certain examples, in accordance with the regimen, the circulating cancer stem cell population, the circulating endothelial cell population and/or the circulating endothelial progenitor population is monitored periodically (e.g., after 2, 5, 10, 20, 30 or more doses of one or more of the therapies or after 2 weeks, 1 month, 2 months, 6 months, 1 year, or more of receiving one or more therapies).

D. Monitoring Treatment

Any in vitro or in vivo (ex vivo) assays known to one of ordinary skill in the art that can detect and/or quantify cancer stem cells can be used to monitor cancer stem cells in order to evaluate the impact of a treatment utilizing a targeted cargo protein. These methods can be used to assess the impact in a research setting as well as in a clinical setting. The results of these assays then may be used to alter the targeting moiety, cargo protein or alter the treatment of a subject. Assays for the identification of cancer stem cells are provided in US patent application no. 2007/0292389 to Stassi et al. (herein incorporated by reference).

Cancer stem cells usually are a subpopulation of tumor cells. Cancer stem cells can be found in biological samples derived from cell culture or from subjects (such as a tumor sample). Various compounds such as water, salts, glycerin, glucose, an antimicrobial agent, paraffin, a chemical stabilizing agent, heparin, an anticoagulant, or a buffering agent can be added to the sample. The sample can include blood, serum, urine, bone marrow or interstitial fluid. In another example, the sample is a tissue sample. In a particular example, the tissue sample is breast, brain, skin, colon, lung, liver, ovarian, pancreatic, prostate, renal, bone or skin tissue. In a specific example, the tissue sample is a biopsy of normal or tumor tissue. The amount of biological sample taken from the subject will vary according to the type of biological sample and the method of detection to be employed. In a particular example, the biological sample is blood, serum, urine, or bone marrow and the amount of blood, serum, urine, or bone marrow taken from the subject is 0.1 mL, 0.5 mL, 1 mL, 5 mL, 8 mL, 10 mL or more. In another example, the biological sample is a tissue and the amount of tissue taken from the subject is less than 10 milligrams, less than 25 milligrams, less than 50 milligrams, less than 1 gram, less than 5 grams, less than 10 grams, less than 50 grams, or less than 100 grams.

A test sample can be a sample derived from a subject that has been treated with a targeted cargo protein. Test samples can also include control samples. In some examples a control sample is from a subject prior to treatment with a targeted cargo protein and in other examples the test sample can be taken from a different location within a subject that has been treated with a targeted cargo protein. Control samples can also be derived from cells that have been artificially cultured. The sample can be subjected to one or more pretreatment steps prior to the detection and/or measurement of the cancer stem cell population in the sample. In certain examples, a biological fluid is pretreated by centrifugation, filtration, precipitation, dialysis, or chromatography, or by a combination of such pretreatment steps. In other examples, a tissue sample is pretreated by freezing, chemical fixation, paraffin embedding, dehydration, permeabilization, or homogenization followed by centrifugation, filtration, precipitation, dialysis, or chromatography, or by a combination of such pretreatment steps. In certain examples, the sample is pretreated by removing cells other than stem cells or cancer stem cells from the sample, or removing debris from the sample prior to the determination of the amount of cancer stem cells in the sample.

In certain examples, the amount of cancer stem cells in a subject or a sample from a subject is/are assessed prior to therapy or regimen to establish a baseline. In other examples the sample is derived from a subject that was treated using a targeted cargo protein. In some examples the sample is taken from the subject at least about 1, 2, 4, 6, 7, 8, 10, 12, 14, 15, 16, 18, 20, 30, 60, 90 days, 6 months, 9 months, 12 months, or >12 months after the subject begins or terminates treatment. In certain examples, the amount of cancer stem cells is assessed after a certain number of doses (e.g., after 2, 5, 10, 20, 30 or more doses of a therapy). In other examples, the amount of cancer stem cells is assessed after 1 week, 2 weeks, 1 month, 2 months, 1 year, 2 years, 3 years, 4 years or more after receiving one or more therapies.

Targets on cancer stem cells are also expressed on normal non-cancerous cells. Therefore, in some examples the identification of cancer stem cells can be made by comparing the relative amount of signal generated from target binding in a control sample and comparing it to the test sample for which the presence or absence of cancer stem cells is being determined. In such examples, the number, quantity, amount or relative amount of cancer stem cells in a sample can be expressed as the percentage of, e.g., overall cells, overall cancerous cells or overall stem cells in the sample.

The results from testing a sample for the presence of cancer stem cells and/or the amount of cancer stem cells present can be used to alter treatment regimes, including altering the variety of targeted cargo protein used. For example, if testing before and after treatment reveals that the population of cancer stem cells increased and/or did not decrease treatment can be altered. For example the dosage of the therapeutic can be altered and/or a targeted cargo protein designed to target distinct target can be substituted or added to the treatment regime.

The amount of cancer stem cells can be monitored/assessed using standard techniques known to one of ordinary skill in the art. Cancer stem cells can be monitored by obtaining a sample, and detecting cancer stem cells in the sample. The amount of cancer stem cells in a sample (which may be expressed as percentages of, e.g., overall cells or overall cancer cells) can be assessed by detecting the expression of antigens on cancer stem cells. Any technique known to those skilled in the art can be used for assessing the population of the cancer stem cells. Antigen expression can be assayed, for example, by immunoassays including, but not limited to, western blots, immunohistochemistry, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, immunofluorescence, protein A immunoassays, flow cytometry, and FACS analysis. In such circumstances, the amount of cancer stem cells in a test sample from a subject may be determined by comparing the results to the amount of stem cells in a reference sample (e.g., a sample from a subject who has no detectable cancer) or to a predetermined reference range, or to the patient him/herself at an earlier time point (e.g., prior to, or during therapy). For the purposes of immunoassays one or more of the targets displayed by the cancer stem cell can be used as the target for the immunoassay.

For example, leukemia stem cells can be identified using a CD34+ target, breast cancer using a CD44+ target, brain a CD133+ target, ovarian a CD44+ target, multiple myeloma a CD19+ target, melanoma a CD20+ target, ependymona a CD133+ target, prostate a CD44+ target, as well as other targets known to be expressed on cancer stem cells. Additional cancer stem cell markers that can be targeted include, but are not limited to, CD123, CLL-1, combinations of SLAMs (signaling lymphocyte activation molecule family receptors) and combinations thereof. Additional exemplary markers can be found in Sakariassen et al., Neoplasia 9(11):882-92, 2007 and Vermeulen et al., Cell. Death Differ. 15(6):947-58, 2008 and U.S. patent application 2008/0118518, which is herein incorporated by reference.

E. Therapeutic Variations

One of ordinary skill in the art will appreciate that targets on cancer stem cells can also be expressed on normal healthy cells. For example, CD133 was initially shown to be expressed on primitive hematopoietic stem and progenitor cells and retinoblastoma and then subsequently shown to be expressed on cancer stem cells. Therefore, in some examples where a cancer stem cell target is expressed on a class of non-cancerous cells therapy can involve removal of a population of the non-cancerous cells followed by targeted cargo protein treatment directed to the cancer stem cell of interest and then reintroducing the non-cancerous cells expressing the target.

In another example, healthy populations of cells that express the same target as that of a cancer stem cell population are protected though the use of two or more targeted cargo proteins. A first targeted cargo protein is engineered to target a first cancer stem cell target (e.g., CD133). The cargo protein that is included in the first targeted cargo protein can be a toxin that is in an inactive form. A second targeted cargo protein is engineered to target a second target on the cancer stem cell (e.g., CD24). This second targeted cargo protein includes a protein sequence capable of activating the first targeted cargo protein. Thus, only a cancer stem cell that expresses the targets for both the first targeted cargo protein and the second cargo protein will receive the therapeutic activity of the cargo moiety.

In another therapeutic variation the subject is treated with an agonist to the target displayed on the cancer stem cell. The cancer stem cells then display an increased level of the target. The treatment with the agonist can then be administered before, during or after administration of the targeted cargo protein. One of ordinary skill in the art will appreciate that the exact timing of administration will depend upon the specific agonist chosen and the specific targeted cargo protein.

### EXAMPLES

### Example 1

This example describes making circularly permuted ligands, such as a ligand specific for a cell-surface receptor found on cancer stem sells. Exemplary ligands include IL-4 and IL-2.

The coding sequence of a chosen ligand is designed to be reorganized creating a new amino termini and a new carboxy termini. The site of reorganization is selected and coding regions are developed synthetically or using the native sequence as a template. PCR can be used to amplify the separate coding regions and the 5′ and 3′ ends of the separate fragments are designed to overlap, thus allowing for the formation of a new coding sequence in which the newly generated peptide can for example encode a first amino acid that in the native protein may have been the 40^{th} amino acid. Specific examples of making circularly permuted ligands are provided in U.S. Pat. No. 6,011,002.

### Example 2

This example describes an in vitro assay that can be used to test the activity of a targeted cargo protein directed to cancer stem cells.

The target that is to be targeted by the targeted cargo protein is recombinantly expressed in a human cell line. Antibodies to the target are used as a positive control for expression and display of the target. Varying concentrations of the targeted cargo protein are contacted with the transformed cells and cell lysis or apoptosis is determined using standard methods.

### Example 3

This example describes in vitro assays that can be used to determine the activity of a targeted cargo protein against cancer stem cells that exist within human brain tumors.

Samples of human brain tumor tissue are washed, mechanically and enzymatically dissociated as described in Reynolds et al. Science 255:1707-1710, 1992 and resuspended in a chemically defined serum-free neural stem cells medium containing growth factors described in Singh et al., Cancer Res. 63: 5821-8, 2003. Cancer stem cells can be identified by their capacity to proliferate, their ability to form colonies or spheres in culture that contain differentiated cells typical of the parent tumor type, and also by their capacity to self-renew, as described in Singh et al., Cancer Res. 63:5821-8, 2003.

Primary sphere forming assay. The tumor cell suspensions are cultured in limiting-dilution cultures with or without various concentrations of a targeted cargo protein, IL4 linked to pseurdomonas toxin (PRX321, shown in FIG. 1). The tumor cells are plated in 96-well microwell plates at cell concentrations ranging from 100 to 10,000 cells per well. Seven days later, the percentage of wells not containing spheres for each cell plating density is determined. Fewer wells containing PRX321 are found to contain spheres than drug-free wells.
Secondary sphere forming assay for self-renewal. Individual spheres from wells in the primary sphere forming assay from wells not containing drug are harvested, dissociated and re-plated in limiting dilutions from 200 down to 1 cell per well in the presence or absence of varying concentrations of PRX321. After 7 days, fewer wells containing PRX321 contain spheres than wells without drug. Cells harvested from spheres are examined for the presence of CD133, a cancer stem cell marker using flow cytometry.
Cell proliferation assay. Tumor cells are plated at a density of 1000 cells/well with and without varying concentrations of PRX321. Cell proliferation is assed on days 0, 3, 5 and 7 post-plating using the Roche 3-(4,5-dimethylthiazol-2-yl)-2,5-dihenyltetazolium bromide-based colorimetric Assay Cell Proliferation kit 1. Quantification of viable cells through reading of UV absorption spectrums at 575 nm are performed on a Versamax microplate reader. There is less cell proliferation in wells containing PRX321 at effective concentrations.
Differentiation assay. Primary tumor cells are cultured for 7 days in the presence and absence of varying concentrations of PRX321. The resulting cells are examined by immunostaining to detect established markers of differentiated neuronal cells as described in Singh et al., Cancer Res. 63:5821-8, 2003.
Sphere forming assay with isolated cancer stem cells. Brain tumor cells are subjected to magnetic bead cell sorting to separate the stem cell and non-stem cell fractions, CD133+ and CD133− respectively as described in Singh et al., Nature 432: 396-401, 2004. The CD133+ cells are plated in the sphere-forming assay described above in limiting dilution in the presence or absence of varying concentrations of PRX321. After 7 days, fewer wells containing drug have spheres than wells without drug, demonstrating inhibition of cancer stem cells by PRX321.

The assays described in this Example for testing the activity of the targeted cargo protein PRX321 (IL4 linked to Pseudomonas toxin) against cancer stem cells from brain may be used to test the activity of other targeted cargo proteins taught herein on other tumor tissues, including, but not limited to, prostate, colon, breast, pancreas and kidney. Other markers for cancer stem cells can be employed in addition to or instead of CD133. For example, colon cancer stem cells are known to express high levels of CD44 and EpCAM (epithelial cell adhesion molecule) as well as CD 166 cell surface markers, and the use of these markers to identify and select or isolate cancer stem cells from colon tumors can be found in see Dalerba et al., PNAS (US.) 104: 10158-10163, 2007. Pancreatic cancer stem cells express high levels of CD44, CD24 and ESA (epithelial-specific antigen) cell surface markers, and such markers may be used to identify and select or isolate cancer stem cells from pancreatic tumors as described in Lee et al., Translational Oncology 1: 14-18, 2008.

### Example 4

This example describes an in vivo assay that can be used to test the activity of a targeted cargo protein when administered locally in a mouse xenograft tumor model.

CD133+ cells (or cells bearing other stem cell markers as described above) are isolated from primary human prostate, brain, breast, kidney, ovarian, melanoma or colon tumors by magnetic bead cell sorting as described by Singh et al., Nature 432: 396-401, 2004, Dalerba et al. (cited above) and Lee et al. (cited above). These cells are injected into individual NOD-SCID (non-obese diabetic, severe combined immunodeficient) mice in numbers ranging from 10² to 10⁴ cells per injection. Tumors resulting in the injected mice are tested for the presence of a subpopulation of CD133+ cells (or cells bearing other cancer stem cell markers) by FACS analysis. Mice with CD133+ cells are treated with targeted cargo proteins that include either a targeting moiety specific for CD133, IL-4R linked to Pseudomonas exotoxin (such as PRX321) or other targeting moiety as taught herein. Targeted cargo proteins are injected intratumorally and systemically at different time points after tumor engraftment. Different doses of targeted cargo proteins range from 100 ng to 10 mg. Tumor growth is assessed 1, 2, 3, 4, 5, 6, 7, 8, 9, 10, 15, 20, and 25 days, and thereafter weekly up to 6 months after treatment. Tumor biopsy samples are assessed histopathologically for the presence of cancer stem cell markers as described above. Mice are also weighed to determine negative effects from treatment. Repeated treatments with targeted cargo proteins may be given over a period of several days. For cancer stem cells isolated from brain, CD133+ cells are injected into the mouse forebrain (see Singh et al., Nature 432: 396-401, 2004). Cancer stem cells from other tumor types are injected into the flank of the mouse, for example, see Dalerba et al. cited above.

### Example 5

This example describes administering a targeted cargo protein to a human to assess toxicity. Additionally, the example describes methods to reduce potential antigenicity.

The intratumoral injection of the targeted cargo proteins described above can demonstrate the usefulness of the targeted cargo protein therapy for localized cancer treatment. However, such a therapy can also be administered by other routes, such as intravenous (iv), intramuscularly, orally, etc., as a systemic therapy for metastatic prostate cancer. However, systemic administration of the targeted cargo proteins disclosed herein may result in the development of a neutralizing antibody response that would limit repeat dosing.

The kinetics and magnitude of the antibody response to any of the targeted cargo proteins disclosed herein can be determined as follows. For example, the antigenic response to IL-4 linked to Pseudomonas exotoxin can be determined in immunocompetent mice, to develop a dosing regimen that can be used in an immunocompetent human. Immunocompetent mice (C57-BL6) are administered iv doses of IL-4 linked to Pseudomonas toxin (PRX321, see U.S. Pat. No. 7,314,632, which is herein incorporated by reference and FIG. 1 using varying regimes such as daily, 5 times a week, weekly, and biweekly at dose ranges of from 0.1 to 5 μg/kg. Mice are sacrificed at varying intervals (e.g., following single dose, following multiple doses) and serum obtained.

An ELISA-based assay can be used to detect presence of anti-pseudomonas toxin antibodies. In this assay, a defined quantity of pseudomonas toxin is fixed to the polystyrene surface in 96-well plates. Following adequate blocking with bovine serum albumin (BSA), serum from mice exposed to the targeted cargo protein is added to the wells at varying dilutions. After a defined incubation time, wells are washed, and alkaline phosphatase linked goat-anti-mouse secondary antibody is added, followed by substrate. The amount of antibody present is determined by measuring absorbance in a spectrophotometer, which permits determination of the time course and magnitude of the antibody response by varying schedules and doses of iv targeted cargo protein.

To decrease antigenicity of the pseudomonas toxin, alternative pseudomonas toxins can be rotated into the regime. The alternative pseudomonas toxins can be generated using random mutagenesis and then tested to ensure they maintain their cytotoxic activity. Another method that can be used to allow continued treatment with prostate-specific protease activated toxins is to use alternative lytic toxins with non-overlapping antigenicity.

### Example 6

This example describes the therapeutic use of a target cargo protein in human subjects with recurrent glioblastoma multiforme (GBM).

PRX 321 is delivered by convection-enhanced delivery (CED) intratumorally. CED is performed by direct infusion through intracranial catheters (1 or more, depending on the size of the tumor) under constant pressure, as described by Patel et al., Neurosurgery 56: 1243-52, 2005, over a period of 1 to 7 days. The total dose of PRX321 is about 90-100 μg, although may be adjusted within the range of range 5 μg to 1 mg. MRI imaging prior to, during and following infusion is used to monitor drug distribution and tumor response. Subjects are monitored by clinical evaluation and MRI on an ongoing basis after treatment.

In view of the many possible embodiments to which the principles of my invention may be applied, it should be recognized that the illustrated embodiments are only preferred examples of the invention and should not be taken as a limitation on the scope of the invention. Rather, the scope of the invention is defined by the following claims. I therefore claim as my invention all that comes within the scope and spirit of these claims.

1. A method of treating a cancer stem cell in a subject, comprising:
administering to the subject a targeted cargo protein, wherein the targeted cargo protein comprises:
(a) one or more cargo moieties; and
(b) one or more targeting moieties that bind to a target displayed by a cancer stem cell, wherein the targeting moiety is derived from a natural ligand to the target, thereby treating the cancer stem cell in the subject.

2. The method of claim 1, wherein the cargo moiety comprises a toxin.

3. The method of claim 2, wherein the toxin comprises a bacterial toxin, animal toxin, or plant toxin.

4. The method of claim 2, wherein the toxin comprises a pore-forming toxin.

5. The method of claim 4, wherein the pore-forming toxin comprises aerolysin or proaerolysin.

6. The method of claim 3, wherein the plant toxin comprises bouganin or ricin.

7. The method of claim 3, wherein the bacterial toxin comprises Pseudomonas exotoxin, cholera toxin, or diphtheria toxin.

8. The method of claim 1, wherein the cargo moiety comprises a pro-apoptosis member of the BCL-2 family selected from BAX, BAD, BAT, BAK, BIK, BOK, BID BIM, BMF and BOK.

9. A method of treating a cancer stem cell in a subject, comprising:
administering to the subject a targeted cargo protein, wherein the targeted cargo protein comprises:
(a) one or more cargo moieties selected from aerolysin, proaerolysin, bouganin, ricin, Pseudomonas exotoxin, cholera toxin, diphtheria toxin, and BAD; and
(b) one or more targeting moieties that bind to a target displayed by a cancer stem cell, thereby treating the cancer stem cell in the subject.

10. The method according to claim 9, wherein the one or more targeting moieties is selected from an antibody, ligand or ligand variant.

11. The method of claim 1, wherein the target displayed by the cancer stem cell comprises a receptor selected from the group consisting of: IL-4, IL-3, IL-2, EGF, and GMCSF or an antigen comprising EpCAM, mesothelin, or CD22.

12. The method of claim 9, wherein the targeting moiety comprises a humanized antibody.

13. The method of claim 1, wherein the targeted cargo protein comprises a human cargo moiety selected from the group consisting of RNase A and perforin.

14. The method of claim 1, wherein the targeted cargo protein comprises a fusion protein.

15. The method of claim 1, wherein the targeted cargo protein is present in a pharmaceutically acceptable carrier.

16. The method of claim 1, wherein the subject has a recurrent cancer or a newly diagnosed cancer.

17. The method of claim 1, wherein the subject is refractory.

18. The method of claim 1, further comprising:
determining whether the subject is refractory to radiation or chemotherapy; wherein if the subject is refractory it indicates that they will benefit from administration of the targeted cargo protein.

19. The method of claim 1, further comprising administering chemotherapy or radiation therapy to the subject after administering the targeted cargo protein, or surgically removing at least part of a tumor after administering the targeted cargo protein.

20. The method of claim 1, further comprising administering chemotherapy or radiation therapy to the subject before administering the targeted cargo protein, or surgically removing at least part of a tumor before administering the targeted cargo protein.

21. The method of claim 1, further comprising administering chemotherapy or radiation therapy to the subject during treatment with the targeted cargo protein, or administering the targeted cargo protein during surgical removal of least part of a tumor in the subject.

22. The method of claim 1, further comprising:
administering to the subject an agonist that sensitizes the cancer stem cells prior to administering the targeted cargo protein.

23. The method of claim 1, wherein the targeted cargo protein is administered intratumorally.

24. The method of claim 1, wherein the targeted cargo protein comprises Pseudomonas exotoxin linked to circularly permuted IL-4, IL-2 linked to aerolysin, IL-2 linked to proaerolysin, IL4 linked to BAD, GMCSF linked to BAD, EGF linked to proaerolysin, anti-EpCAM antibody linked to Pseudomonas exotoxin, anti-EpCAM antibody linked to bouganin, anti-mesothelin antibody linked to PE, anti-CD22 antibody linked to PE, anti-CD22 antibody linked to RNase A, and anti-PSMA antibody linked to thapsigargin.

25. The method of claim 1, further comprising removing hematopoietic stem cells from the subject prior to administering the targeted cargo protein.

26. The method of claim 25, further comprising re-introducing to the subject the removed hematopoietic stem cells.

27. The method of claim 1, wherein the method further treats a bulk tumor in the subject.

28. The method of claim 1, wherein the targeted cargo protein further comprises a polymer, for example to increase stability, increase circulating half life or reduce immunogenicity of the targeted cargo protein.


SEQUENCE LISTING (1 sequence)

SEQ ID NO: 1 - protein, 485 aa, Artificial Sequence
  targeted cargo protein
MDTTEKETFCRAATVLRQFYSHHEKDTRCLGATAQQFHRHKQLIRFLKLRDRNLWGLAGLNSCPVKEANQSTLENFLERLKTIMREKYSKCSSGGNGGHKCDITLQEIIKTLNSLTEQKTLCTELTVTDIFAASKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL